(19) United States
(12) Patent Application Publication
(10) Pub. No.: US 2007/0275019 A9
(43) Pub. Date: Nov. 29, 2007

(54) Vaccines directed to cancer-associated carbohydrate antigens

(76) Inventors: Sen-Itiroh Hakomori, Mercer Island, WA (US); Kazuko Handa, Bellevue, WA (US)

Correspondence Address: BELL, BOYD & LLOYD, LLP, P.O. Box 1135, CHICAGO, IL, 60690, US

(21) Appl. No.: 10/669,264

(22) Filed: Sep. 25, 2003

Related U.S. Application Data
(63) Continuation of application No. 10/040,336, filed on Jan. 9, 2002, now abandoned.
Application No. 10/040,336 is a continuation of application No. 09/696,213, filed on Oct. 26, 2000, now abandoned.
Application No. 09/696,213 is a continuation of application No. 09/253,024, filed on Feb. 19, 1999, now abandoned.

(51) Int. Cl. A61K 39/00; A61K 31/737

(52) U.S. Cl. 424/277.1; 514/54

(57) ABSTRACT

A vaccine and method to prevent or to retard the growth and replication of cancer cells that express a carbohydrate wherein the vaccine comprises: (a) a pharmaceutically effective amount of a carbohydrate antigen found on said cancer cells, or a mimetic thereof; and (b) a pharmaceutically acceptable carrier. The carbohydrate antigen can be Tn or sialyl-Tn.

### FIELD OF THE INVENTION

The instant invention relates to materials and methods of active immunization to cancer cell carbohydrate epitopes. Various synthetic antigens can be used to elicit an immune response to cancer cells expressing those antigens.

### BACKGROUND OF THE INVENTION

Synthesis of sugar chains of glycoproteins and glycolipids often is blocked in oncogenic transformation (Hakomori & Murakami, Proc. Natl. Acad. Sci. USA, 59:254-261, 1968 and Hakomori, Cancer Res., 45:2405-2414, 1985). Thus a number of cell surface molecules with short carbohydrate chains and without peripheral structures, i.e., without modification of the core structure, are found in cancer and precancer states. For example, common core structures of mucin-type glycoproteins present in normal tissues in a cryptic form (Springer, Science, 224:1198-1206, 1984 and Hirohashi et al., Proc. Natl. Acad. Sci. USA, 82:7039-7043, 1985) are revealed in cancer and precancer states, such as the Tn and sialyl-Tn antigens.

### SUMMARY OF THE INVENTION

Accordingly, it is an object of the instant invention to provide materials and methods of vaccine development, i.e., of preventing or retarding growth and replication of cancer cells by administering antigens that can induce antibodies or other immune responses specific for carbohydrate antigens expressed by the cancer cells.

The vaccine comprises:

(a) a pharmaceutically effective amount of an antigen which induces antibodies and other cellular immune responses to carbohydrate determinants expressed on cancer cells, and

(b) a pharmaceutically acceptable carrier, such as a bacterial adjuvant or a chemically synthesized adjuvant.

In another embodiment, the antigen of part (a) is replaced with an antigen mimetic.

The method comprises inducing an anti-cancer cell immune response by administering the above-described vaccine to a subject.

The instant invention describes the chemical synthesis of polymeric Tn or sialyl-Tn antigen or of a lactone of same. The antigen is conjugated to a carrier, such as keyhole limpet hemocyanin. The instant invention teaches the use of a chemically synthesized core mucin as an immunogen to prevent or to retard the growth of tumors expressing Tn or sialyl-Tn.

Furthermore, the instant invention describes the chemical synthesis of (i) tandemly linked Tn or sialyl-Tn antigen; or (ii) mimetics of Tn or sialyl-Tn antigen, including peptide mimetics selected out of a phage display random peptide library as well as stabilized, modified forms thereof, lactone or lactam forms of sialyl-Tn, or other modified forms of Tn or sialyl-Tn which induce an immune response to Tn or sialyl-Tn. Antigens, either type (i) or (ii) as above, can be linked directly to a dendrinmeric multivalent core, which then can be linked to a known carrier. The carrier preferably should have binding affinity to polypeptide chains of type 1 or type 2 major histocompatibility complex (MHC) proteins. Alternatively, a suitable carrier is one which is a protein macromolecule known in the art or a chemically synthesized core mucin.

### BRIEF DESCRIPTION OF THE FIGURES

FIGS. 1A and 1B are schematic representations of the chemical synthesis of the carbohydrate epitopes Tn and sialyl-Tn conjugated to carrier molecules as described in the examples. Synthesis of Tn (α-GalNAc linked to the hydroxyl group of serine (Ser), threonine (Thr) or any other compound) can be accomplished chemically although the enzymatic synthesis of Tn also is possible. Synthesis of sialyl-Tn could readily be achieved by adding a sialyl residue to the 6-hydroxyl group of α-GalNAc (Tn) enzymatically or chemically.

FIGS. 2A-C describe methods of making vaccines, including schemes of synthesizing antigen clusters with tandemly linked Tn or sialyl-Tn (A), constructing multivalent systems (B) and configuring appropriate vaccines (C). For a description of symbols and abbreviations see the examples below. A cluster of Tn or sialyl-Tn can be assembled on tandemly linked Ser-Ser-Ser or Ser-Thr-Thr oligopeptides or an oligopeptide having another order of any number of Ser and Thr residues (usually three is a suitable number). A cluster of Tn or sialyl-Tn also can be assembled by tandemly linking same to multiple hydroxyl or amino groups in other structures with suitable distance and orientation, that is, similar to that of tandemly linked residues of Ser or Thr in Ser-Ser-Ser or Ser-Thr-Thr, or another order of any number of Ser and Thr residues.

FIG. 3 describes a scheme for synthesizing derivatized serine antigen cluster 5 of FIG. 2A.

FIG. 4 describes a scheme for synthesizing derivatized Tn or sialyl-Tn on tandemly linked Ser or Thr, cluster 6 of FIG. 2A.

FIGS. 5A and 5B describe a scheme for preparing the core structure comprising lysine residues and a spacer arm, and conjugating said core structure with derivatized serine.

FIGS. 6A and 6B describe schemes for conjugating antigen clusters with synthetic lipopeptide or liposaccharide (non-macromolecular) carriers.

FIGS. 7A and 7B describe vaccines comprising lipid carrier, peptides and antigen.

FIG. 8 depicts a scheme for preparing dimeric Tn antigen.

FIGS. 9A and 9B depict a structure for tandemly linked Tn or sialyl-Tn on Ser-Thr-Thr (FIG. 9A) and on other residues (R) having similar distance and orientation as Ser and Thr in FIG. 9A. Such a structure with a spacer (X) having functional groups ready to link to a carrier molecule is shown in FIG. 9B.

FIG. 10 presents a general scheme for selection of a defined peptide or peptides having complementarity to any compound affixed to a solid phase (for example, plastic surface). The selection is made using a phage display random peptide library. To select peptides having the same surface profile as a defined carbohydrate antigen or lipid antigen, the surface is coated with a specific anti-carbohydrate or anti-lipid antibody. To the coated surface is added the phage display random peptide library (step A). Unbound phage-peptide is washed out and only bound phage-peptide is selected (step B). The phage is used to infect E. coli (step C) which is propagated (step D) to yield amplified phage-peptide (step E). The cycle is repeated 4 or 5 times. The number of phage-peptides initially applied for one selection experiment could be 5×10⁷ to 10⁸. Specific phage carrying specific peptides are recovered (step F).

FIG. 11A and its legend illustrate the generally-accepted mechanism to induce T cell-dependent B cell proliferation leading to antibody (mainly IgG) production. The mechanism occurs through antigen-presenting cells (APC) bearing MHC class II proteins which bind complementary peptides and then present the peptides to helper T (CD4⁺) cells. Exogenously-injected antigen initially is taken up by APC's, which may be macrophages, dendritic cells in skin, B cells etc. Antigens are taken up and processed within those cells. Those peptides having binding affinity to MHC class II proteins are presented by the APC to CD4⁺ cells through the TCR/CD3 complex. In that mechanism, CD4 is essential to recognize part of the α chain of the MHC class II proteins. The process stimulates CD4 cells to proliferate, leading to production of cytokine, particularly IL-2. Thus, CD4 cell proliferation is enhanced and leads to stimulation of B cells through binding of processed peptide to Ig receptor (IgR). Specific binding of peptide to MHC α or β proteins is essential, and such a peptide is useful for stimulating an anti-carbohydrate antibody response when a carbohydrate epitope is presented through such a peptide. When the peptide fragment held by the MHC class II protein is presented to TCR/CD3 in a CD4⁺ cell, a weak accessory binding takes place between the APC and CD4 cells, for example, mediated by the binding of ICAM1 to LFA1, LFA3 to CD2, and B7 to CD28.

FIG. 11B illustrates restricted presentation of peptides through MHC class I protein to cytotoxic CD8⁺ cells. Many types of cells bearing MHC class I proteins are capable of processing endogenously synthesized peptide and presenting the peptide to CD8⁺ killer T cells. The peptide is recognized by the TCR/CD3 complex and by CD8, resulting in stimulation of CD8⁺ cell proliferation, which causes lysis of antigen-bearing target cells. Specific binding of peptide to MHC class I α protein is essential. If a specific carbohydrate linked to a defined peptide which is capable of binding to MHC class I α protein occurs, a T cell immune response directed to the carbohydrate antigen may occur. When a peptide fragment is presented to the TCR/CD3 complex in a CD8⁺ cell, a weak accessory binding takes place analogous to that between the APC and CD4 cell as above, that is, involvement of ICAM1, LFA1 etc.

FIG. 12 presents a scheme explaining the importance of defining a peptide or peptides having binding capability to MHC class II and I proteins as provided in FIGS. 11A and B. The processed peptide has surface compatibility with MHC class I α chain (panel A), which is shown in enlarged view in the bottom part of the panel. The peptide processed through the APC is presented through MHC class II α and β chains (panel B), as shown in enlarged view in the bottom part of the panel. In panel A, MHC class I molecules hold peptides consisting of 8 to 111 (usually 9) amino acids, tightly fixed in the peptide binding groove. A few HLA allele-specific peptide consensus sequences are indicated at the top of the diagram. Class II ligands, consisting of 12-25 amino acid residues, are not tightly fixed by their ends in the groove, but are allowed to hang out. To induce killer T cell response directed to carbohydrate epitopes, the epitope should be held by peptides having a consensus sequence. In contrast, a specific peptide sequence capable of binding to class II proteins is a useful carrier to induce helper T cell responses directed to carbohydrate epitopes. The scheme is adapted and modified from Hammer et al., J. Exp. Med. 176: 1007, 1992, and from Ramensee & Monaco, Curr. Opin. Immunol. 6: 1, 1994.

FIG. 13 presents a scheme illustrating a combination of two selection procedures based on the use of a phage display random peptide library: (i) for selection of peptides having the same surface profile as Tn or sialyl-Tn; and (ii) for selection of peptides having complementarity to MHC class I or II proteins. The selection procedure is analogous to that shown in FIG. 10.

### DETAILED DESCRIPTION OF THE INVENTION

For the purposes of the instant invention, the following terms have the meanings set forth below.

Mucin-Type Glycoprotein—A high molecular weight protein (Mᵣ>10⁶) with a high degree of O-linked glycosylation at serine or threonine residues. Mucin-type glycoproteins can be polymerized further by S—S-dependent linkage and are the major components of epithelial secretions.

Core Structure of Mucin-Type Glycoprotein—Basic carbohydrate structure without peripheral substitution, a backbone and which is linked directly to the protein moiety of a mucin glycoprotein. The most frequent and the major such structure on cells is the T antigen. The core structures—T, Tn and sialyl-Tn—are common in all types of mucin glycoproteins irrespective of species (various animals and man).

T Antigen—A disaccharide consisting of one mole each of galactose (Gal) and N-acetyl galactose (GalNAc) with a structure as follows: Galβ1→3GalNAc. The reducing terminal of GalNAc often is α-linked to the hydroxyl group of a serine or threonine residue of a polypeptide chain or any hydroxyl group on another molecule. On the other hand, the disaccharide can be linked to any carrier using known chemical linkers and techniques.

Tn Antigen—An antigen wherein the innermost GalNAc residue can be α-linked directly to the hydroxyl group of a serine or threonine residue of a polypeptide chain at the cell surface. Other hydroxyl group containing compounds can serve as a carrier. Linkers can be used to attach Tn to carriers not bearing hydroxyl groups. Since α-GalNAc is part of the blood group A antigen, many anti-Tn antibodies cross-react with A antigen.

Sialyl-Tn Antigen—An antigen wherein the sixth hydroxyl group of the α-GalNAc residue of the Tn antigen is substituted with sialic acid (also known as N-acetyl neuraminic acid, NeuAc), i.e., NeuAcα2→6GalNAc. The antigen is α-linked to the hydroxyl group of a serine or threonine residue of a polypeptide chain at the cell surface. Other compounds containing a hydroxyl group can be used as the backbone to which the sialyl-Tn is bound. Linkers can be used to attach sialyl-Tn to carriers not containing a hydroxyl group using known chemistries.

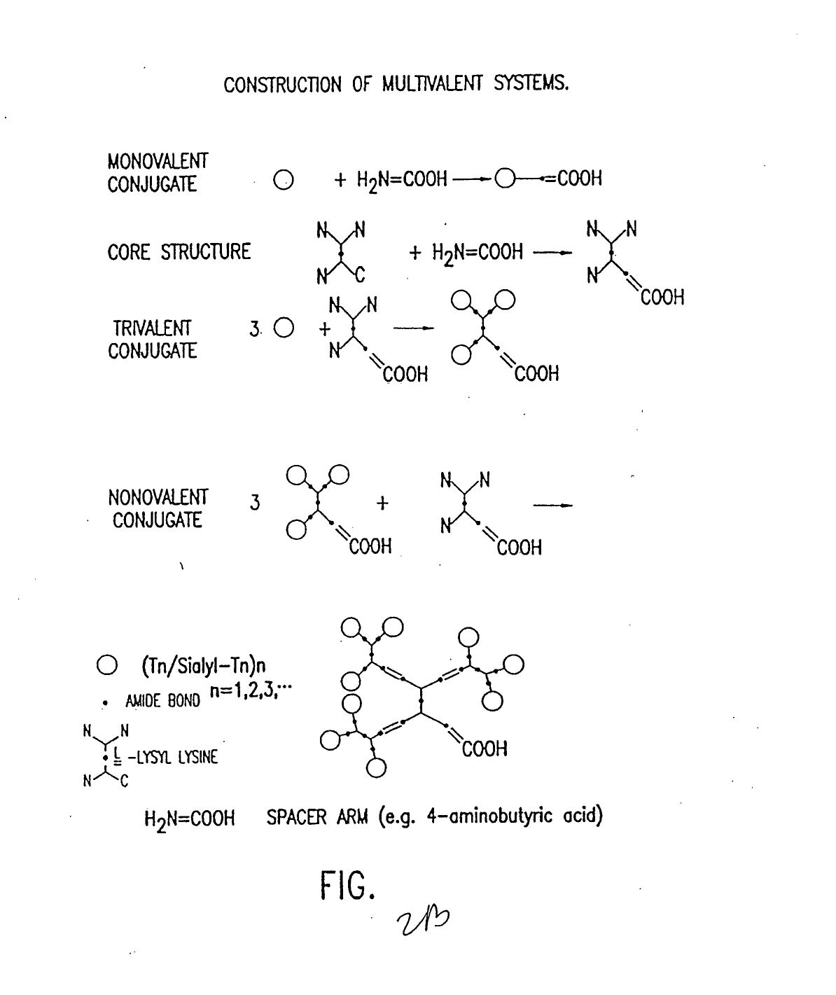

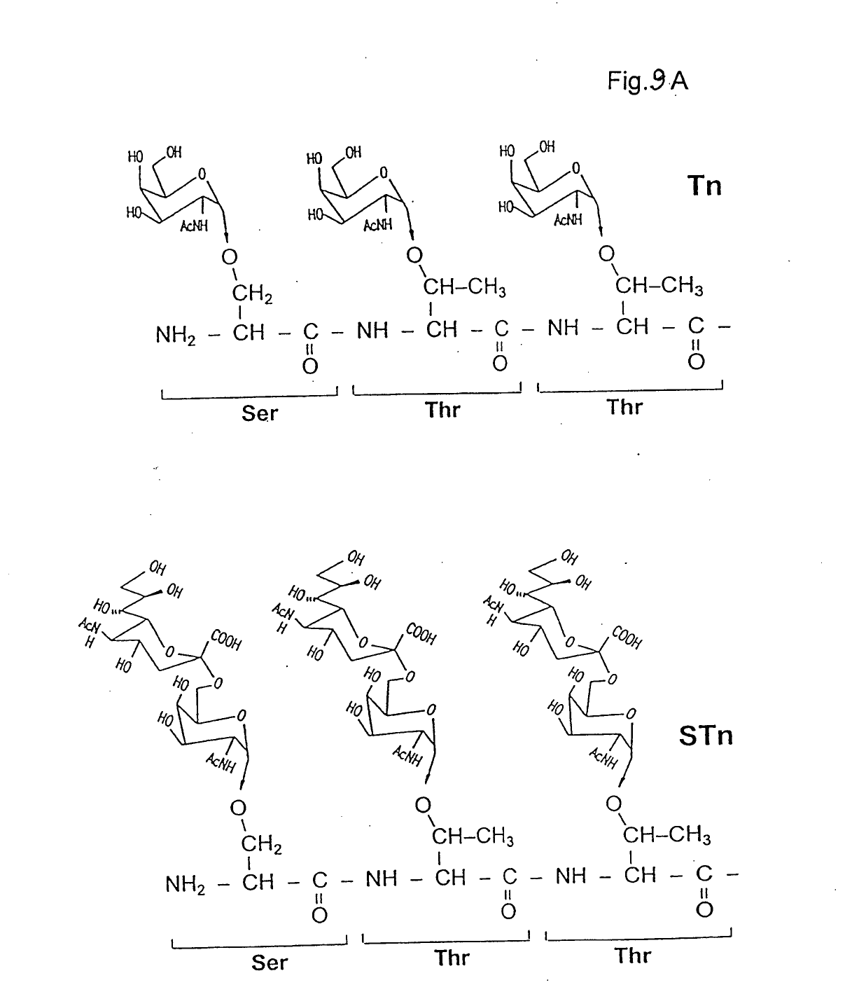

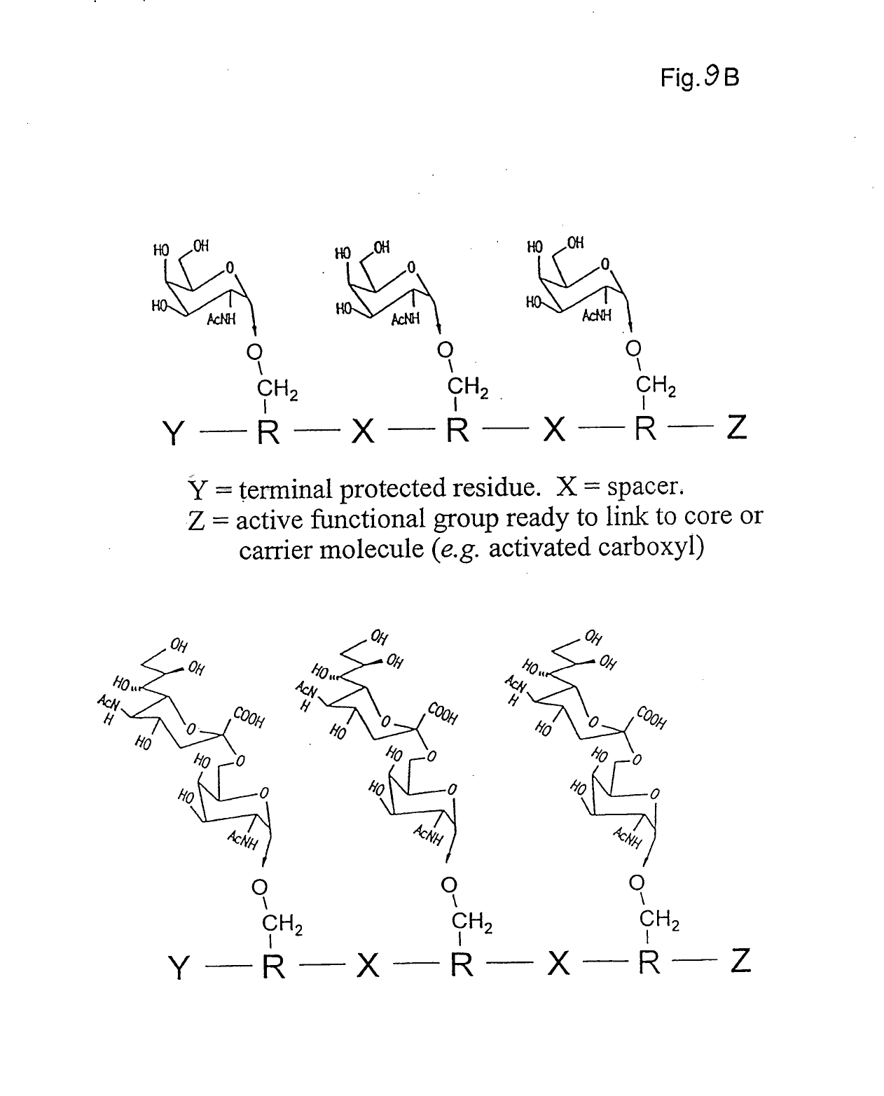

Tandemly-linked Tn or sialyl-Tn antigen—Tn or sialyl-Tn antigen defined as above is linked to every hydroxyl group of Ser-Ser-Ser, Ser-Thr-Thr, or another order or number of Ser and Thr residues (three is a preferable number) in an oligopeptide. A cluster of Tn or sialyl-Tn also can be assembled by tandem linking to multiple hydroxyl or amino groups in other structures with suitable distance and orientation, as described in the legend of FIGS. 2A-C and in FIG. 9.

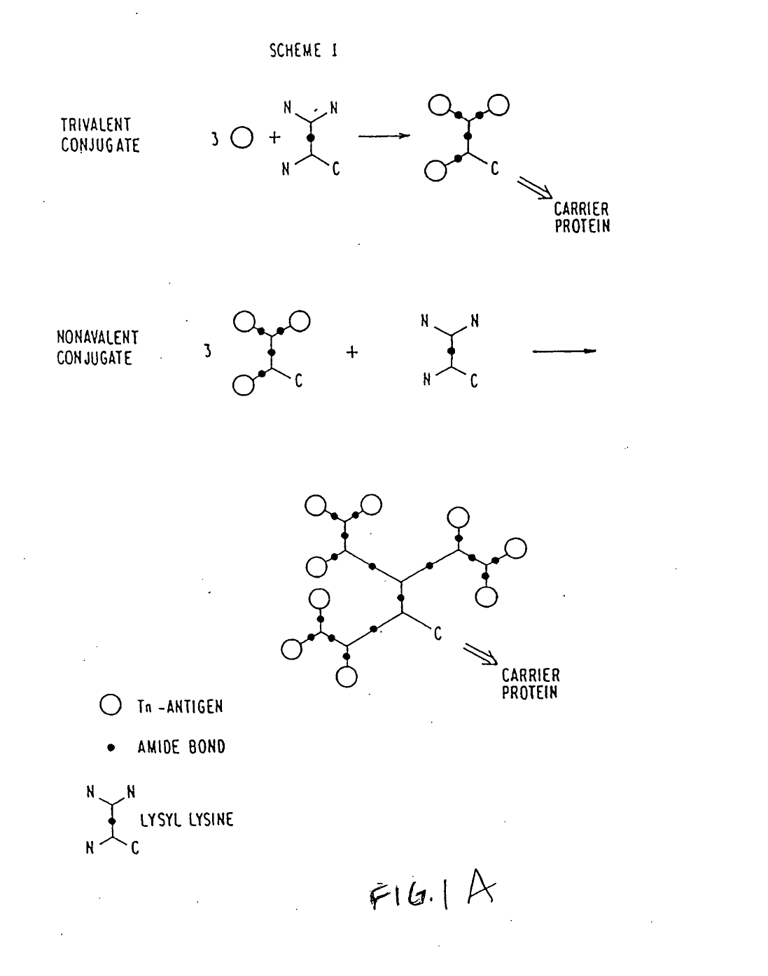

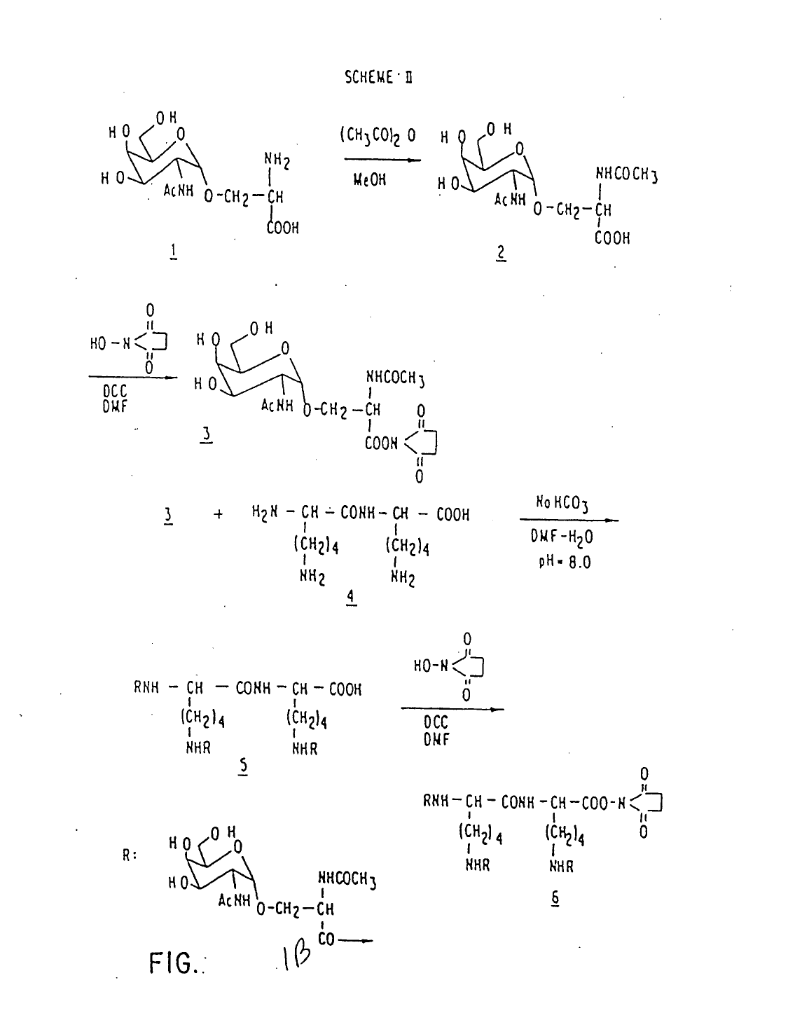

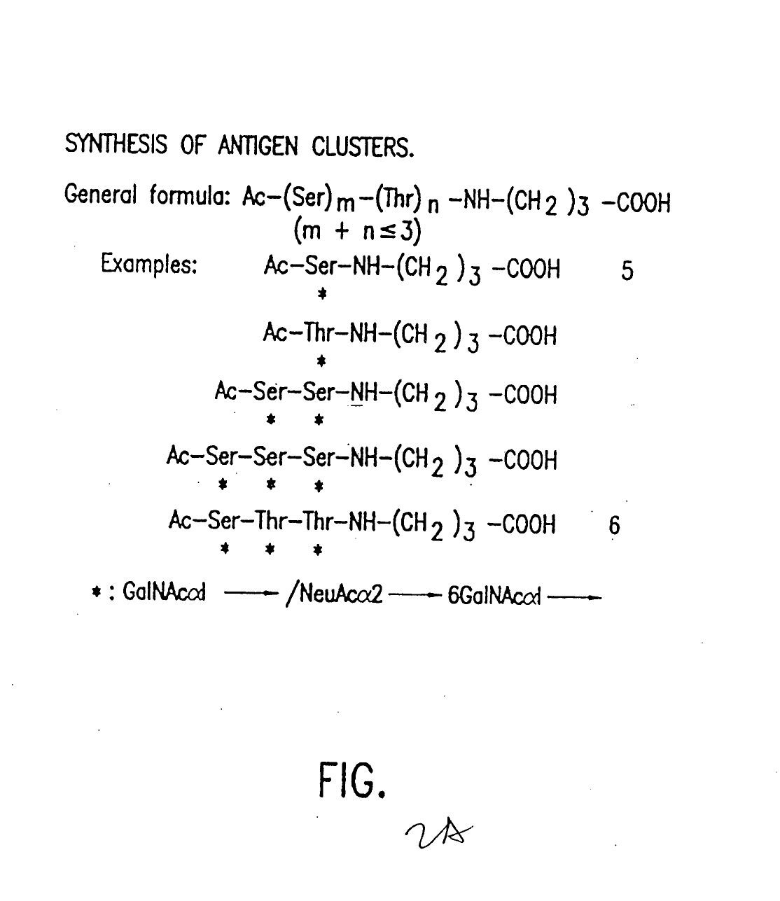

Multivalent Tn or sialyl-Tn antigen—Tn or sialyl-Tn antigen defined as above, or tandemly linked Tn or sialyl-Tn antigen defined as above, are linked to a multivalent functional group, typically carried by a dendrimeric core structure, for example, as described by Tarn (Proc. Natl. Acad. Sci. USA 85: 5409-13, 1988); see FIGS. 1A and 2B.

Carrier Protein or Carrier Peptide—Tandemly linked Tn or sialyl-Tn antigen, or multivalent Tn or sialyl-Tn are bound to a carrier protein, such as, the oft-used keyhole limpet hemocyanin (KLH) to induce an immune response. One example is shown in FIG. 1A in which Tn antigen is carried by a dendrimer core and one carboxyl group of the dendrimer is linked to a carrier protein. Other examples are shown in FIG. 2B in which tandemly linked Tn or sialyl-Tn is carried by a dendrimer and one carboxyl group of the dendrimer is designed to bind to a carrier protein. For an anti-cancer vaccine, an IgG or a T cell response is desirable. Activation of helper T cells, which occurs through presentation of carbohydrate antigen or its mimetics by a peptide or peptides having binding capability to MHC class II proteins, is desirable.

Mimetics—Slightly modified epitopes often display stronger immunogenicity than naturally-occurring epitopes. A typical example is a lactone of the sialyl α2→3Gal residue present in various gangliosides. The lactone structure contains an additional 6-membered ring between the carboxyl group of the sialyl residue and the 2-hydroxyl group of penultimate Gal. Sialyl-Tn has the sialyl residue at the α2→6GalNAc position and therefore, the lactone structure may not be stable. Therefore, other strategies may be need to be considered to stabilize a lactone of sialyl-Tn. On the other hand, modification of sialyl-Tn can include a sialyl α2→6 N-methyl GalNAc group or an N-formyl GalNAc group might be considered as alternative strategies.

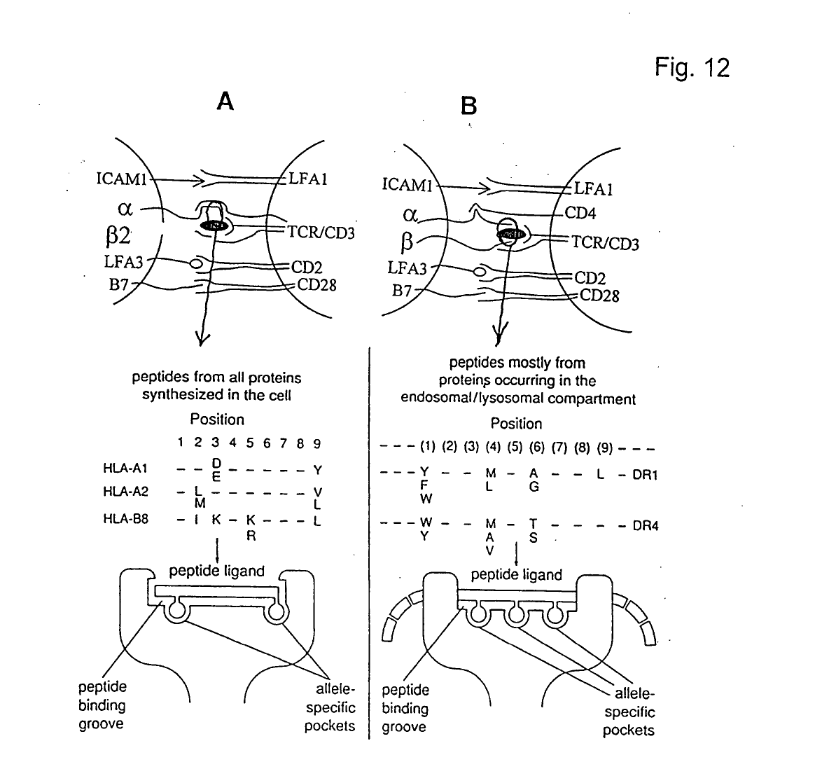

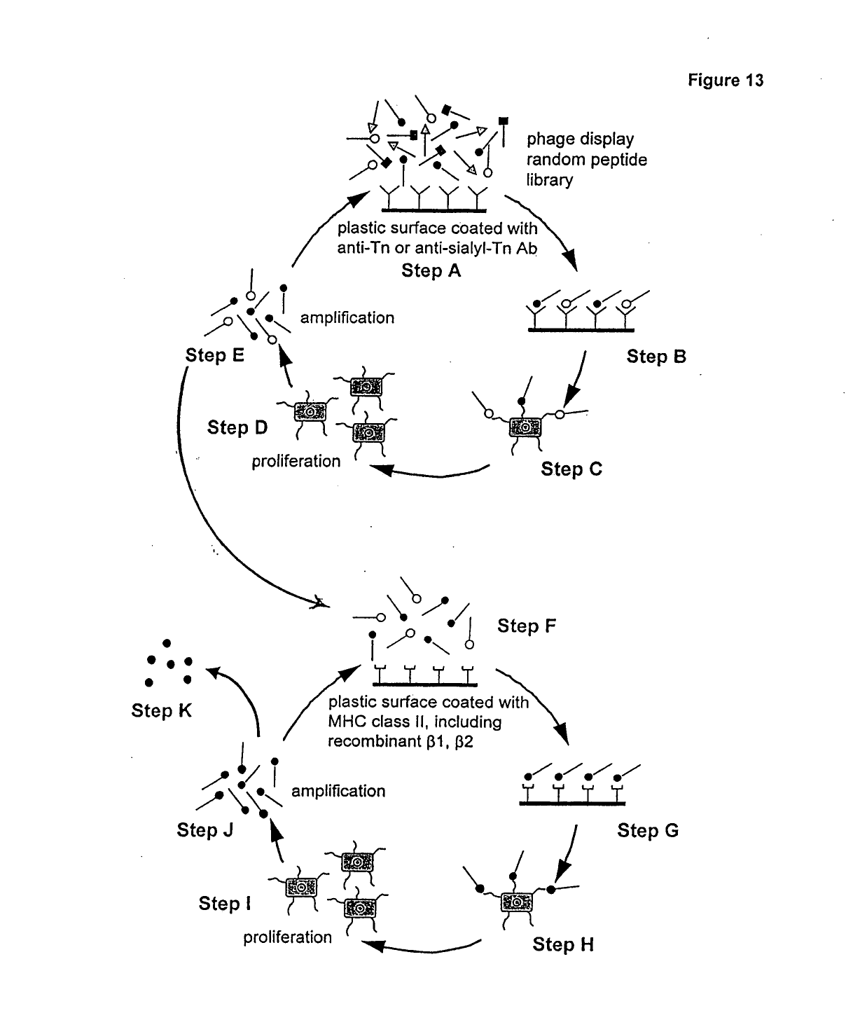

Peptide mimetics are another important approach to obtain a defined peptide sequence having a surface structure similar to that of Tn or sialyl-Tn. Such peptide mimetics can be selected from a phage display random peptide library as illustrated in FIGS. 12 and 13. The peptide mimetics are stabilized by appropriate modification using known chemistries.

Mucin-type Glycoproteins—naturally occurring structures expressing, for example, T, Tn or sialyl-Tn, can be obtained by enzymatic or chemical modification of, for example, a mucin-type glycoprotein to expose a core structure or by isolation of mucins having core structures. Such mucins are present in some animal species.

Examples of types of enzymatic modifications that can be used to expose the core structure of various mucin-type glycoproteins include the elimination of the terminally located α2→3 sialyl residue by influenza virus sialidase or the total elimination of all sialic acid residues by Clostridium perfringens sialidase. Enzymatic modification also can include treatment with β-galactosidase (preferably from Charonia lampas), α-fucosidase and N-acetylhexosamidase. Enzymatic hydrolysis of mucin glycoprotein is described by Hirohashi et al. (Proc. Natl. Acad. Sci. USA, 82:7039-7043, 1985).

Examples of chemical reactions which can be used to expose the core structure of mucin-type glycoproteins include periodate oxidation followed by reduction with sodium borohydride and treatment with weak acid. The procedure is called Smith degradation (Spiro, Meth. Enz., 28:3-43, 1972). The chemical treatment eliminates the non-reducing terminals of carbohydrate residues except sialic acid, which can be eliminated by sialidase treatment, as described above.

Examples of mucins isolated from animals that can be used as immunogens include ovine submaxillary mucin (OSM) in which 90% of the carbohydrate chains consist of the sialyl-Tn antigen and bovine submaxillary mucin (BSM) in which 50% of the carbohydrate chains consist of the sialyl-Tn antigen and 30% of the carbohydrate chains consist of Tn antigen and other unidentified residues. Not all the structures of the mucin glycoproteins of animal species have been elucidated; however, novel structures such as the trihexosamine core (GlcNAcβ1→4[GlcNAcβ1→3]GalNAc), which was previously found in sheep gastric mucin (Hounsell et al., Biochem. Biophys. Res. Commun., 92:1143-1159, 1979), may well be present in some of the core structures of human cancer mucin and, if so, can be used in the instant invention. Systematic knowledge of mucin core structures of various animals species is incomplete, however, as the mucin core structures become known, one skilled in the art readily will be able to determine if such are useful in the instant invention.

Additionally, on further systematic application with various animal species, common structures as immunogens might be found. Methods to elucidate such structures include alkaline hydrolysis in the presence of sodium borohydride (β-elimination), methylation analysis and mass spectrometry of each oligosaccharide liberated. The methods are compiled in Hakomori & Kannagi, 1986, in “Handbook of Experimental Immunology”, Vol. I, Blackwell Scientific Publications, Oxford, pp. 9.1-9.39.

For example, mucin-type glycoproteins which will be modified enzymatically or chemically to produce core structures can be isolated by gel filtration through SEPHAROSE 4B (bead-formed matrix of agarose, trademark of and distributed by Pharmacia, Piscataway, N.J.) or SEPHACRYL 200S (bead-formed matrix of acrylamide, distributed by Pharmacia).

The isolated glycoprotein then is modified enzymatically or chemically by, for example, methods described above, to expose the core structure. The core structure can be purified, for example, by gel filtration through SEPHAROSE 4B or SEPHACRYL 200. High pressure chromatography on a synthetic molecular filter column (fast liquid chromatography (FPLC), Pharmacia) also is useful to separate enzymatically or chemically modified mucins. However, as immunogen, the modified mucin does not need to be purified so long as unwanted side reactivity is not observed or does not cause undesirable side effects.

Mucins that are derived from animal species and contain glycoproteins already in the form of a core structure are obtained by conventional methods. For example by gel filtration through SEPHAROSE 4B, SEPHACRYL 200, or FPLC, as described above.

As mentioned above, especially preferred antigens are the Tn antigen and the sialyl-Tn antigen.

Tn antigen can be prepared from any type of glycoprotein by successive treatment with exoglycosidases and β-galactosidase. The latter enzyme must be able to cleave the β1-3galactosyl structure linked to α-GalNAc.

Examples of suitable exoglycosidases and β-galactosidases include sialidase from Clostridium perfringens (Sigma Chemical Co., St. Louis, Mo.) and β-galactosidase of Charonia lampas (Seikagaku Kogyo, Tokyo, Japan).

The successive treatment with exoglycosidase and β-galactosidase is carried out as follows. A stable solution of mucin in suitable buffer containing 0.02% of a suitable detergent is mixed with enzyme and incubated at 37° C. for several to 18 hours. A suitable buffer is 50 mM acetate, pH 4.5-5.0, containing 0.02-0.05% TRITON X-100 or NP-40 (Both are non-ionic detergents. TRITON is comprised of octylphenoxy polyethoxy ethanol and other surface active compounds and is a registered trademark of Rohm & Haas. NONIDET P-40 or NP-40 is comprised of octylphenol-ethylene oxide condensate containing an average of 9 moles of ethylene oxide per mole of phenol, distributed by Sigma) often is used.

The thus treated glycoprotein then is purified for use as an immunogen by gel filtration with an appropriate column as described previously. The purification of immunogen is not essential, since the presence of unmodified mucin generally does not interfere with immune response to the modified mucin in vivo.

In addition, some animal mucins, such as ovine submaxillary mucin, contain a large quantity of Tn antigen in its native form, but the majority is sialylated. Therefore, sialidase-treated ovine submaxillary mucin is an excellent immunogen to elicit a Tn immune response. Another efficient Tn immunogen is a native Tn glycoprotein secreted from cells, such as the human squamous cell lung carcinoma cell lines QG56 and LU65 (Hirohashi et al., Proc. Natl. Acad. Sci. USA, 82:7039-7043, 1985), or human hepatoma cell line HUH7.

The cells are cultured in suspension and the spent culture medium is lyophilized to reduce the volume to about 1/50 of the original volume. The concentrated spent medium then is dialyzed. The dialyzed material is placed on SEPHAROSE 4B and gel filtered. The void volume is pooled, concentrated further and re-chromatographed on SEPHACRYL 200. The glycoprotein fraction in the void volume is used as immunogen.

To obtain Tn or sialyl-Tn antigen from cultured cells, the procedure is as described above.

A better source is ovine submaxillary mucin as described above. Ovine submaxillary mucin is obtained from ovine submaxillary gland and purified by known techniques.

The carbohydrate epitopes, Tn and sialyl-Tn, can be synthesized chemically and covalently linked to synthetic or natural carriers such as polylysine, human serum albumin or highly branched synthetic carrier molecules based on the tert-butoxycarbonyl β-alanine unit such as described by Tam (Proc. Natl. Acad. Sci. USA, 85:5409-5413, 1988). Other means of conjugation which do not alter significantly the expression of Tn or sialyl-Tn can be used.

Antigen Design—for cancer immunotherapy or vaccine development, an IgG response or a T cell response is desirable, although it is not uncommon for carbohydrate antigens to generate an IgM response. To overcome difficulties in achieving those goals, the carbohydrate antigens are designed to maximize immunogenicity and the ability to stimulate a variety of elements of the immune response. Carbohydrate antigens preferably are tandemly linked. To enhance epitope density, the tandemly linked antigen can be multivalently assembled. Mimetics with slightly modified structures (for example, lactones or N-modified amino sugars) or peptide mirnetics are assembled. Such peptide mimetics can be selected from a phage display random peptide library. The tandemly linked, multivalently assembled antigen or mimetic epitopes are bound to carrier proteins, peptides, lipopeptides or lipids, for example. The carrier can be a specific peptide selected for affinity to the major histocompatibility complex (MHC) class II or class I proteins. The peptide carrier can be selected from a phage display random, peptide library as well.

The carbohydrate epitope Tn can be synthesized chemically by established procedures (Grundler & Schmidt, 1984, Liebigs. Ann. Chem. 1826, 1984; and Toyokuni et al., Bioorg. Med. Chem. 2: 1119-32, 1994). Enzymatic synthesis of Tn using Ser-Ser-Ser, Ser-Thr-Thr or another order of Ser and Thr residues (generally three is a suitable number) also is possible by α-GalNAc transferase specific for said peptide (Bennett et al., J. Biol. Chem. 271: 17006-12, 1996; Wandall et al., J. Biol. Chem. 272: 23503-14, 1997).

The epitope, sialyl-Tn, can be synthesized chemically by established procedures for sialylation of Tn, for example, the Tn synthesized as above can be α2→6 sialylated by a specific sialyltransferase (Kurosawa et al., J. Biol. Chem. 269: 1402-9, 1994). The epitopes, tandemly linked together as shown in FIG. 2A and FIG. 9, can be (i) linked directly to a “carrier protein” or a defined “carrier peptide” having specific affinity to MHC class II protein; or (ii) linked to a multivalent dendrimer based on the tert-butoxycarbonyl β-alanine unit, as described by Tam (Proc. Natl. Acad. Sci. USA 85: 5409-13, 1988), which then, for example, is linked to a “carrier protein” or a “carrier peptide” as above.

The instant invention provides a vaccine and method for preventing or retarding growth and replication of cancer cells and a medicament and method for treating cancer.

More specifically, the instant invention provides a vaccine for preventing or retarding growth and replication of cancer cells that express carbohydrate antigens, such as Tn and sialyl-Tn.

The vaccine comprises:

(a) a pharmaceutically effective amount of an antigen, expressed on cancer cells, conjugated to a carrier macromolecule, peptide, lipopeptide or liposaccharide, and

(b) a pharmaceutically acceptable carrier including bacterial or chemically synthesized adjuvant.

Alternatively, the vaccine comprises:

(a′) a pharmaceutically effective amount of an antigen mimetic, where the original or cognate antigen is expressed on cancer cells; and

(b′) a pharmaceutically effective amount of synthetic carrier polypeptide to which the carbohydrate epitope or its mimetic is bound. The carrier peptide has an affinity to MHC (either class II or class I). Such carrier peptide or peptides can be selected from a phage display random peptide library.

Similarly, the method comprises inducing an anti-cancer cell immune response by administering to a subject the above-described vaccine.

The anti-cancer cell immune response can be produced by antibodies directed against the carbohydrate determinant or by inducing various other types of immune responses such as induction of helper T cells, cytotoxic killer T cells, anomalous killer cells (AK cells), antibody dependent cytotoxic cells, NK cells etc.

Induction of Antibody Response—antibodies induced by tandemly-linked Tn or sialyl-Tn have a high binding affinity to naturally-occurring Tn or sialyl-Tn antigen expressed on the tumor cell surface. Anti-sialyl-Tn antibody TKH2 (Kjeldsen et al., Cancer Res. 48: 2214-20, 1988) and anti-Tn antibody CU1 (Takahashi et al., Cancer Res. 48: 4361-7, 1988) have properties similar to those of the BM series of antibodies described in U.S. Pat. No. 5,660,834. Synthetic Tn or sialyl-Tn antigen therefore are designed preferably with a tandemly-linked structure and bound to an appropriate carrier molecule. As an example, tandemly-linked Tn antigen bound to lipopeptide as carrier was found to elicit strong IgG and IgM responses (Toyokuni et al., J. Am. Chem. Soc. 116: 395-6, 1994; Bioorg. Med. Chem. 2: 1119-32, 1994) and to suppress growth of Tn-expressing TA3Ha mouse mammary carcinoma, see Example 5 hereinbelow.

Tandemly-linked carbohydrate epitopes are qualitatively different from randomly-linked carbohydrate, as observed typically for Tn or sialyl-Tn. Multivalent structures, when the same carbohydrate residue is linked to the end of a branched core structure such as a Starburst dendrimer, may induce enhanced immunogenicity. Such an assembly for Tn and sialyl-Tn, as well as their tandemly-linked structures, is explained in FIGS. 1 through 4. Assembly of a multivalent core also is important for the lipid carrier, as explained in FIGS. 6 through 8. Multivalent dendrimers based on the tert-butoxycarbonyl β-alanine unit are explained above.

Doses, methods of administrating and suitable pharmaceutically acceptable carriers can be determined readily by the skilled artisan. For example, some of the parameters can be extrapolated from dose-response studies in animals and particularly non-human primates. Currently, clinical trials are underway on the use of sialyl-Tn in the treatment of breast cancer.

In a preferred embodiment, injection of cyclophosphamide, an inhibitor of suppressor T-cell response, is included as part of the administration routine, as is commonly known in the art.

Suitable carriers are lipids such as certain adjuvants and compounds that stimulate T-cells. Examples include Freund's adjuvant, Ribi adjuvant and BCG.

Ribi adjuvant is an adjuvant essentially composed of trehalose dimycolate and monophosphoryl lipid A, the effective component of mycobacteria known to stimulate the immune response of T-cells as well as B-cells (Ribi et al., Clin. Immunol. Newsletter, 6:33-36, 1985). Because Ribi adjuvant is also a T-cell stimulator, it is an especially preferred carrier. Additionally, the carrier can be the carrier macromolecule used to prepare the chemically synthesized mucin-type glycoproteins described above.

Another way to obtain an enhanced immune response is to derivatize the antigen. One approach is to generate lactones.

Lactones are defined, for example, as the inner ester between the carboxyl group of sialic acid and the primary and secondary hydroxyl group of a sugar residue within the same molecule. One example is GM₃ lactone, wherein the carboxyl group of sialic acid is esterified with the C-2 secondary hydroxyl group of the penultimate galactose (Yu et al., J. Biochem. Tokyo, 98:1307 (1985)). The structure is sterically stable and relatively stable at acidic to neutral pH, although unstable at alkaline pH.

Lactones can be prepared by dissolving an antigen in glacial acetic acid and allowing the solution to stand for at least 48 hours, followed by lyophilization of the acetic acid. Formation of the lactones can be monitored by thin layer chromatography, using high performance thin layer chromatography plates obtained from J. T. Baker Chemical Co. (Phillipsburg, N.J.) and, for example, chloroform:methanol:water (50:40:10 (v/v/v)) containing 0/05% (w/v) CaCl₂ as a solvent since lactones can show a distinctively higher mobility than native gangliosides on thin layer chromatography. The above solvent composition is not critical and any well known solvent which can separate parent molecules from the lactones thereof can be employed, for example, as described in Nores, G. A. et al., J. Immunol., 139:3171-3176 (1987).

Alternatively, and more efficiently, lactones can be prepared by dissolving a carbohydrate in chloroform:methane:12 N HCl (10:35:4.5 (v/v/v)) and allowing the solution to stand for about one day. The resulting solution then is chromatographed using DEAE-Sephadex in chloroform:methanol:water (0.1:1:1 (v/v/v)). Two main components and several minor components are resolvable in that system. The resulting lactones can be purified by HPLC on latrobeads 6RS8010 in isopropanol:hexane:water (55:25:30 (v/v/v)) with gradient elution being carried out as described in Watanabe et al. J. Lipid Res., 22:1020-1024 (1981). The structure of the purified lactones can be verified by direct probe fast atom bombardment mass spectrometry as described in Riboni, J. Biol. Chem., 261:8514-8519 (1986).

Other suitable derivatized antigens can be used so long as the immune response generated thereby is specific for Tn or sialyl-Tn. For example, α2→6 N-methyl gal-Nac or N-formyl GaINac may be employed. Also, the derivative instead could be, for example, a mimic of Tn or sialyl Tn made with carbohydrates other than sialic acid and GalNAc. In fact, the mimic need not be a carbohydrate but can be made with other biological molecules, such as with amino acids.

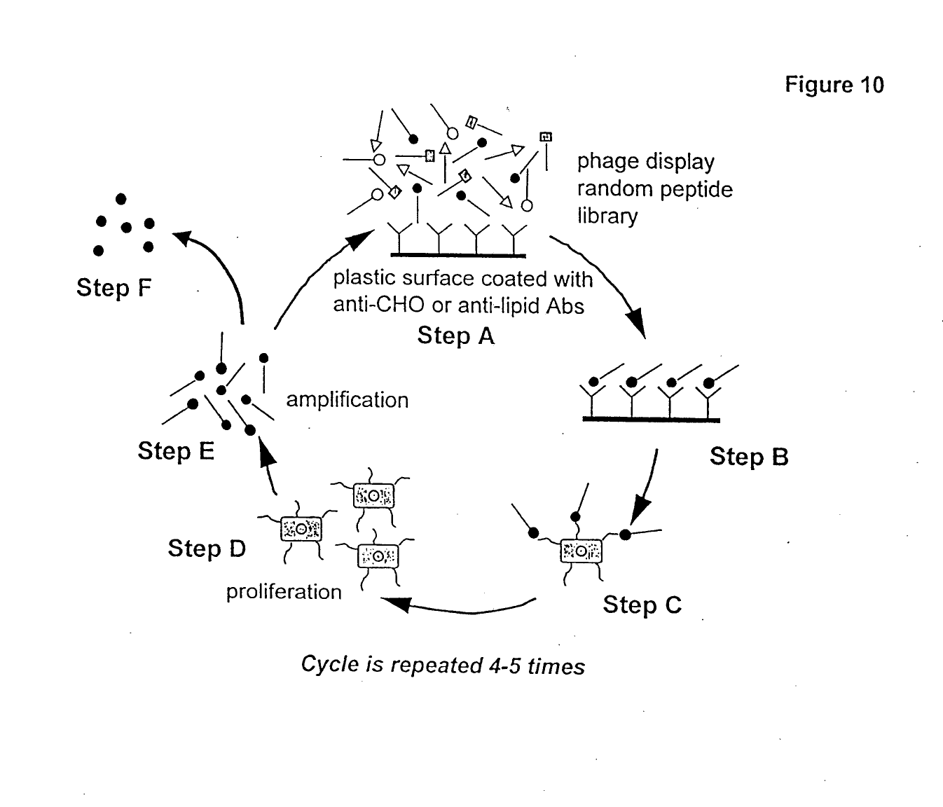

Peptide Mimetics of Carbohydrate Antigens—each antigen has a specific surface profile that is recognized by antibody. A phage display random peptide library has been applied to select peptides that bind to specific protein epitopes (Scott & Smith, Science 249: 386-90, 1990; Devlin et al., Science 249: 404-6, 1990; Cwirla et al., Proc. Natl. Acad. Sci. USA 87: 6378-82, 1990). The principle is shown in FIG. 10. The approach also has been applied successfully to select peptides that mimic specific carbohydrate antigens as deduced by antibody binding (Hoess et al., Gene 156: 27-31, 1995). Using an antibody to Le^{y} (Fucα1→2Galβ1→4[Fucα1→3]GlcNAcβ1→3Galβ1→R) as primer, the peptide, Ala-Pro-Trp-Leu-Tyr-Gly-Pro-Ala, was selected from a phage display random peptide library. Only that peptide inhibited binding of the antibody to Le^{y}.

Specific peptides having defined sequence capable of binding to Tn or sialyl-Tn can be readily selected by anti-Tn antibody or anti-sialyl-Tn antibody coated on a plastic surface. The peptide should be capable of inhibiting antibody binding to Tn or sialyl-Tn antigen. The peptide sequence is verified and which amino acid or acids essential for binding to antibodies is determined. A three-dimensional model is constructed and if necessary, proper modification to stabilize the conformation of the peptide is made to obtain a stable peptide mimetic of a carbohydrate epitope. Such mimetics should be immunogenic to induce Tn or sialyl-Tn antibody response when the peptide is properly linked to a carrier molecule or tandemly linked to a core and then linked to a carrier molecule. Empirically, peptides are better antigens than carbohydrates in terms of inducing an IgG response or a T cell response.

Also, while Tn and sialyl-Tn often are found conjugated to protein via the hydroxyl group of serine or threonine, the Tn or sialyl-Tn epitope may be conjugated to any of a variety of carriers, naturally occurring or synthetic, by any known means. Thus, the carrier can be a protein, where the Tn or sialyl Tn epitopes are bound to residues other than serine or threonine, a lipid and so on.

For cancer immunotherapy or vaccine development, a cellular response such as a T cell immune response in addition to an antibody response is highly desirable. For example, because the carbohydrate determinants are of limited size, one approach to enhancing immune responsiveness is to increase the density of the epitope of interest, as provided herein.

Specific Carrier Which Binds MHC—an alternative approach is to provide the epitope on a more immunogenic carrier, as it is known that carriers can be non-specific activators of the immune system. Thus, the carrier can act as an adjuvant.

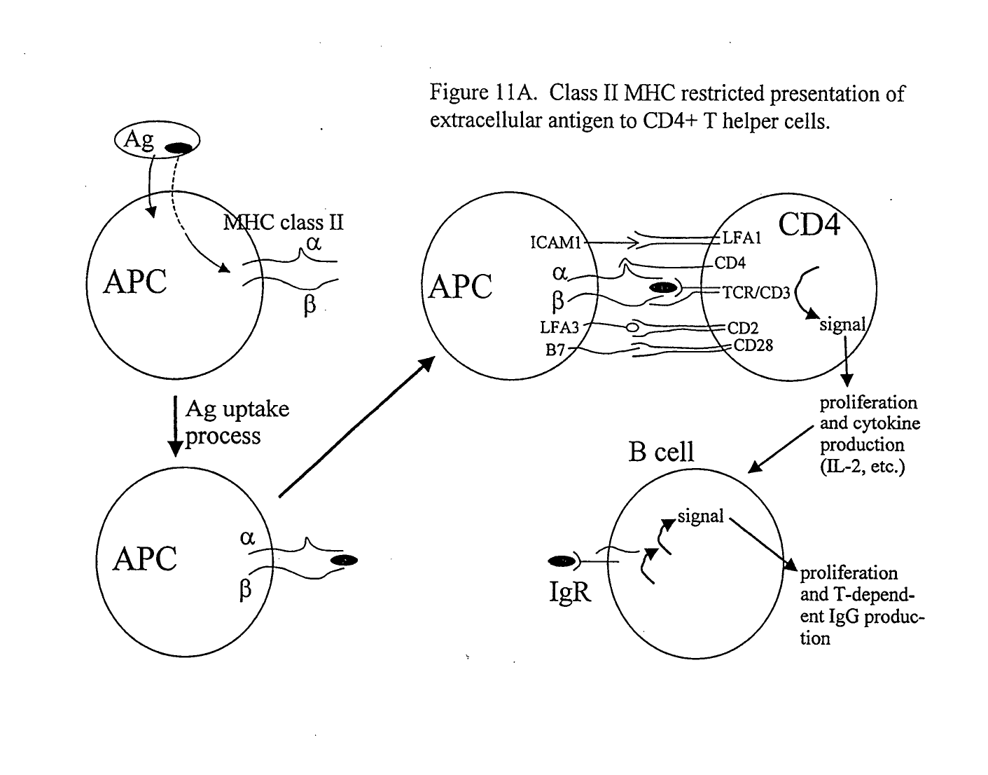

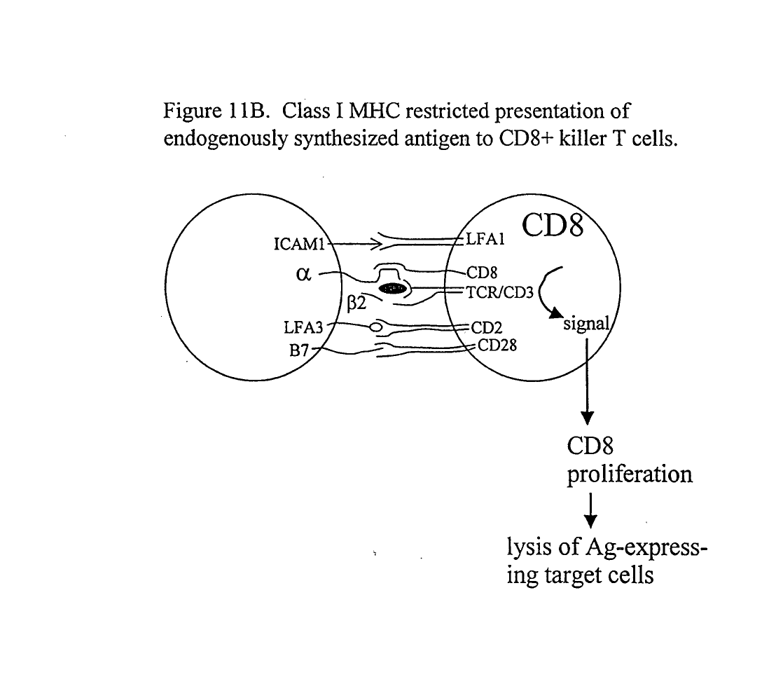

Since anti-Tn and anti-sialyl-Tn antibodies are often IgG rather than IgM, such mucin-type glycoproteins can be processed by antigen-presenting cells (APC). Major Histocompatibility Complex (MHC) class II proteins play a major role in presenting glycopeptide fragments to helper T cells (CD4⁺) to cause CD4⁺ cell proliferation, leading to cytokine production. Cytokines, particularly IL-2, further stimulate CD4⁺ cell proliferation and present glycopeptide to B cells to induce B cell proliferation. The entire process therefore consists of helper T cell-dependent B cell proliferation and production of IgG antibodies (FIG. 11A). Specific peptide sequences that bind to MHC class II proteins are usually 12-25 amino acid residues in size.

About 65-90% of Caucasian, Chinese and Japanese populations, and 55-75% of Hispanic populations have MHC class II DR β1 and β2 proteins (Imanishi et al., “Proc. 11th Intl. Histocompatibility Workshop and Conference”, Vol. 1, Oxford Univ. Press, pp. 1065-74, 1992). Therefore, recombinant MHC class II DR β1 and β2 proteins and portions thereof can be used with confidence to select specific peptides capable of binding to those MHC structures. Peptides are held in the peptide binding groove by interaction with the peptide backbone as well as with some side chains, including carbohydrates, as shown in FIG. 11B.

Selection of such peptides can be made using a phage display random peptide library by the same principle shown in FIG. 10, adapted as in FIG. 13. Such peptides could be useful carriers for tandemly linked Tn or sialyl-Tn to induce a T cell-dependent IgG response.

A specific carbohydrate epitope linked covalently to a defined amino acid residue of a peptide or peptides which bind specifically to MHC Class I region molecules may induce efficient T cell response to the carbohydrate. For example, two peptides that bind to MHC Class I Kb, FAPGNYPAL (derived from vesicular stomatitis virus) and RGYVYQGL (derived from Sendai virus) covalently linked to various oligosaccharides, were tested by immunization with complete Freund's adjuvant. Di-Gal (Galα1-4Gal) oligosaccharide linked to N or P of FAPGNYPAL, or to V or Q of RGYVYQGL, elicited a cytotoxic T cell response to the di-Gal residue, which specifically killed tumor cells bearing the residue or coated with di-Gal glycolipid, in an MHC Class I-unrestricted manner. GM3, GM3-lactam or lactose residue bound at the same position did not induce a T cell response to GM3, GM3-lactam or lactose.

A similar approach using two peptides, ASNENMETM (derived from influenza A virus) and SGPSNTPPEI (derived from adenovirus), that bind specifically to MHC Class I Db, linked covalently to di-Gal or to other above oligosaccharides, did not elicit a T cell response (Abdel-Motal et al., Eur. J. Immunol. 26:544-551, 1996).

Thus, carbohydrates, if properly presented by a defined peptide that fits with an appropriate MHC Class I subtype, may evoke a T cell response. The carbohydrate residue should be of suitable size and placed at a suitable location (middle) of the peptide. The combination of type of carbohydrate structure and structure of binding peptide to MHC Class I may govern the magnitude and specificity of the response, see FIG. 10.

A peptide sequence that binds to various MHC Class I subtypes, in mouse and human, is preferred. Such a common peptide with promiscuous binding ability would be more useful as a carrier of Tn or sialyl-Tn. Such a peptide could be selected by application of a phage display method in a fashion analogous to that used to obtain peptides that bind to the MHC Class II proteins. The methods will allow selection of peptide mimetics simulating Tn or sialyl-Tn.

The principle of the method is based on production of a large number of randomly synthesized peptides (10⁷-10⁸ from one run), some mimicking the surface structure of a given carbohydrate, peptide or lipid. A plasmid bearing a peptide with a surface structure complementary to the given compound is selected and propagated, and the selection process is repeated. In the case of Tn or sialyl-Tn, the plasmid mimetic peptide to be selected mimics the surface structure of Tn or sialyl-Tn itself. Viral peptides that bind to human MHC Class I are known. Nevertheless, a search for new peptide sequences with promiscuous binding ability is desirable.

Regarding T cell immunity, there is a novel T cell population in mice having T cell receptors (TCR) with the V region 14 of the α chain, which develops outside the thymus and shows natural killer activity. The single invariant T cell receptor is called V alpha 14 and the natural killer cell population is called V alpha14 NKT. Knockout mouse experiments showed that V alpha 14 NKT is an essential target of the tumor-suppressive effect of IL-12 (Cui et al., Science 278:1623-1626, 1997).

In contrast to regular cytotoxic T cells, V alpha 14 NKT cells recognize the antigen associated with the CD1d molecule. The synthetic glycosphingolipid, alpha-GalCer, having C26 fatty acid and C18 sphingosine, was found to bind V alpha 14 TCR and to stimulate proliferation of V alpha 14 NKT cells (Kawano et al., Science 278:1626-9, 1997). Treatment in mice bearing various tumors with a GalCer abrogated tumor growth and metastasis, e.g. for B16 melanoma and mouse colonic carcinoma Colon 26 (Nakagawa et al. Cancer Res. 58:1202-7, 1998; study by Tanguichi et al. cover study by McDonald in Jpn. J. Cancer Res. 88(1), 1997).

The CD1b molecule presents lipid antigen to T cells in glucose monomicolate (Moody et al., Science 278:283-6, 1997) and there is a large hydrophobic binding groove for antigen presentation in CD1.

Thus, glycosphingolipid ligands that bind to the invariant human TCR associated with NKT cells, analogous to V alpha 14 TCR, may stimulate proliferation of the NKT cell population in human. Micolyl glycosphinogolipid analogs having various hydrophobic heads, including tumor-associated antigens such as Tn and sialyl-Tn, may be useful in stimulating a specific cellular response.

Pharmaceutical formulations of the instant invention can be of solid form including tablets, capsules, pills, bulk or unit dose powders and granules but preferably are of liquid form including solutions, fluid emulsions, fluid suspensions, semisolids and the like. In addition to the active ingredients, the formulation would comprise suitable art-recognized diluents, carriers, fillers, binders, emulsifiers, surfactants, water-soluble vehicles, buffers solubilizers and preservatives.

Methods of treatment include those known in the art for administering biologically active agents. Such methods include in vivo and ex vivo modalities. For example, an antigen-containing solution can be delivered intravenously, by a pump means attached to a reservoir containing bulk quantities of said solution, by passive diffusion from an implant, such as a Silastic implant, and the like.

The skilled artisan can determine the most efficacious and therapeutic means for effecting treatment practicing the instant invention. Reference also can be made to any of numerous authorities and references including, for example, “Goodman & Gilman's The Pharmaceutical Basis of Therapeutics” (6th ed., Goodman et al., eds., MacMillan Publ. Co., NY, 1980).

The invention will now be described by reference to specific examples. However, the invention is not to be construed as being limited to the examples.

Unless otherwise specified, all percents, ratios etc. are by weight.

### EXAMPLE 1

Preparation of Tn Antigen

Tn antigen was prepared from culture supernatant from human lung squamous cell carcinoma LU-65 (available from the American Type Culture Collection, Rockville, Md.) by gel filtration as described in Hirohashi et al. (Proc. Natl. Acad. Sci. USA, 82:7039-7043, 1985).

Specifically, the cells were cultured in RPMI medium supplemented with 15% fetal calf serum. (However, the cells can also be cultured in other media, e.g., Dulbecco's modified Eagle's medium, and under certain conditions, cells can be cultured in chemically-defined media without supplementation of serum.)

Next, 500 ml of the supernatant obtained by centrifugation to separate cell debris was lyophilized to 1/10 of its original volume, dialyzed against distilled water, and re-lyophilized. The residue was dissolved in 10 ml of PBS; a small part of the residue was insoluble in PBS. Aliquots of 5 ml were applied to a column of SEPHAROSE-CL4B, previously equilibrated with PBS in the presence of 0.1% sodium azide, and elution was performed with phosphate-buffered saline (PBS), i.e., 25-30 mM NaH₂PO₄—Na₂HPO₄ in 0.9% NaCl at pH 7.0. Fractions of 5.0 ml were collected, and aliquots of 100 μl from each fraction were placed in each well in 96-well plastic plates (Falcon, Microtest III, flexible assay plate, Falcon Labware, Oxnard, Calif.), and incubated at room temperature overnight in order to effect efficient adsorption of mucin-type glycoprotein on the plastic plate. Each plate was washed three times with PBS, and 150 μl of 1% BSA in PBS was added to each well. The plates were placed at room temperature (25° C.) for 2 hours to allow blocking of the uncovered plastic surface, i.e., to avoid non-specific adsorption of primary antibody to the uncovered plastic surface. Each plate was again washed three times with PBS and 100 μl of anti-Tn antibody, NCC-LU-81 (diluted 1:1000), were added to each well. The plates were placed at 4° C. for 18 hours to allow antigen-antibody complexes to form. The plates were again washed three times with PBS, and 50 μl of a secondary antibody (rabbit anti-mouse IgM and IgG) diluted 1:1000 with 1% BSA in PBS, were added to each well. The plates were incubated for 2 hours at 25° C. and washed three times with PBS. The secondary antibodies were purchased from Cappel Laboratories (Cochranville, Pa.). Finally, to each well was added 50 μl of ¹²⁵I-labeled protein A having an approximate activity of 10⁵ cpm. The plates were incubated for 90 minutes at room temperature. The plates were washed three times with PBS and the radioactivity in each well was counted in a gamma counter to determine which fractions had the Tn activity. The fractions that contained the Tn activity (fractions 8-15) (Panel A, FIG. 1) were pooled and lyophilized to ⅕ of the original volume, and the sample of 1.0 ml was applied to a column of SEPHACRYL S-200 (1.2×110 cm). The sample was eluted with PBS, pH 7.0 and fractions of 2.0 ml were collected. Aliquots of 100 μl from each fraction were analyzed for protein concentration by UV absorption at 280 nm, and analyzed by solid-phase radioimmunoassay (RIA) for the Tn activity as described above for the SEPHAROSE-derived fractions. Only the highly active fractions at the void volume (Vₒ) were taken (see Panel B, FIG. 1), dialyzed extensively against distilled water, lyophilized, weighed, and used for immunization.

### EXAMPLE 2

Production of Sialyl-Tn Antigen

Ovine submaxillary mucin (OSM) was used as the source of the sialyl-Tn antigen. Approximately 90% of the carbohydrate chains of OSM consist of sialyl-Tn antigen.

OSM was isolated from ovine submaxillary glands by conventional methods. (Tettamanti & Pigman, Arch. Biochem. Biophys., 124:45-50, 1968).

Briefly, an aqueous extract of submaxillary glands was precipitated at acidic pH (e.g., 3.5). This is called a mucin clot. The mucin clot was centrifuged, dissolved in water, the pH adjusted to neutral, and fractional ethanol precipitation in sodium acetate was performed.

### EXAMPLE 3

Inhibition of Syngeneic Tumor Growth in Mice by Immunization with Mucin Glycoprotein Containing Tn Epitope

TA3Ha (Friberg, J. Natl. Canc. Inst. 48:1463, 1972; Van den Eijden et al., J. Biol. Chem. 254:12153, 1979) is an extremely aggressive mouse mammary carcinoma cell line. Intraperitoneal inoculation of as few as 10 TA3Ha cells into CAF₁ mice causes death within 15-20 days. Inoculation of 10³ cells causes death generally within 10-12 days. The results have been obtained repeatedly and provide a baseline of TA3Ha tumor cell malignant potential.

TA3Ha tumor cells have been characterized by expression of T and Tn antigens, defined respectively by monoclonal antibodies (mAb's) HH8 and BM8 (IgG₂ₐ) or LCC-LU35 or -81 (Hirohashi et al., Proc. Natl. Acad. Sci. USA 82:7039-7043, 1985). An attempt was made to suppress growth of that highly malignant tumor line by active immunization of CAF₁ mice with desialylated OSM (asialo-OSM or A-OSM; expresses mainly Tn antigen), or desialylated bovine submaxillary mucin (asialo-BSM or A-BSM; expresses mainly Tn antigen. Immunization of CAF₁ mice with various amounts of A-OSM or A-BSM without Freund's adjuvant failed to suppress growth of TA3Ha tumor cells.

Ovine submaxillary mucin (OSM) containing Tn antigen was purified by the procedures described by Hill et al. (Hill et al., J. Biol. Chem. 252:3791-3798, 1977). Ovine submaxillary glands were homogenized in 0.01 M NaCl. The supernatant was adjusted to pH 4.7, and the precipitate was removed. The supernatant was applied to a sulphopropyl-SEPHADEX C-25 column, and the fractions containing Tn and sialyl-Tn antigens detected by monoclonal antibodies were combined. Mucin was precipitated by addition of acetyltrimethylammonium bromide and centrifuged. The precipitate was redissolved in 4.5 M CaCl₂ and absolute ethanol to a concentration of 60%. The precipitate was discarded, and the supernatant was brought to 75% ethanol. Mucin was collected by centrifugation at 27,000 g for 30 min. The precipitate was dispersed in 1 M NaCl and dialyzed against 10 mM sodium phosphate, pH 6.8. The mucin was applied to an hydroxylapatite column, and fractions containing Tn and sialyl-Tn activity were collected. Bovine submaxillary mucin (BSM) was purchased from Sigma.

OSM and BSM were treated with neuraminidase (from Clostridium perfringens) to produce A-OSM and A-BSM and then applied to a SEPHAROSE CL-4B column. Fractions containing Tn activity were pooled, dialyzed and lyophilized.

Female CAF₁ mice were immunized intraperitoneally (I.P.) with either PBS, Freund's adjuvant (FA) or A-BSM (20 μg) emulsified with complete FA. One week later mice were reimmunized I.P. with PBS, incomplete FA or A-BSM (40 μg) emulsified with incomplete FA. Ten days after the second immunization, mice were challenged with syngeneic mammary tumor TA3Ha cells (10⁴ cells, injected subcutaneously). Tumor size, survival of mice and antibody production were monitored in the mice.

Generally mice in the control FA group died by 14 days after the tumor challenge. On the other hand, mice in the group immunized with BSM were alive 19 days after the challenge.

The BSM-immunized group of mice had a high titer of antibodies in sera specific to Tn and sialyl-Tn, whereas no antibodies to T epitope (neuraminidase-treated glycophorin A) were detected. No antibody titers to Tn antigen were detected in the PBS- or FA-immunized animals.

Tumors grew in both PBS-immunized and BSM-immunized mice, however, the rate of tumor growth was retarded in the BSM-immunized group as compared with the PBS-immunized group.

Tumor Growth Suppression by Vaccination with Tn Antigen and Ribi Adjuvant

Tumor growth suppression was tested using A-OSM or A-BSM with Ribi adjuvant, which is essentially composed of trehalose dimycolate and monophosphoryl Lipid A, the effective component of mycobacteria known to stimulate immune response of T as well as B cells (Ribi et al., Clin. Immunol. Newsletter 6:33-36, 1985). A-OSM and A-BSM were produced by treatment with neuraminidase as described above. Cyclophosphamide (CP), which is known to be an effective inhibitor of suppressor T cell response, also was injected in combination with the antigen/Ribi complex. For example, 100 μg of A-OSM or A-BSM were mixed with 500 μg of complete Ribi adjuvant and injected subcutaneously at day −7 (i.e., 7 days before tumor cell inoculation). On day 0, 700 TA3Ha cells were injected intraperitoneally. On day 1, CP (75 mg/kg) was injected intraperitoneally. On days 2, 5, 12, and 19, 100 μg antigen/500 μg Ribi adjuvant complex was injected subcutaneously. Another experimental group was injected with CP-Ribi on days 2, 5, 12, and 19, respectively. Another experimental group receiving CP only showed some tumor growth suppression, but most animals had died by 20-30 days. In striking contrast, animals receiving repeated inoculations with antigen/Ribi plus CP had significantly longer survival, i.e., in the groups receiving A-OSM/Ribi/CP or A-BSM/Ribi/CP, 50% of the animals lived past day 50.

Acquired Resistance of Immunized Mice to Further Tumor Inoculation

Mice surviving after active immunization with either A-OSM or A-BSM followed by TA3Ha tumor inoculation became highly resistant to further inoculation with increased numbers of the same tumor cells without further active immunization. As noted previously, non-immunized mice died from inoculation with 10³ TA3Ha cells within 12 days. However, surviving immunized animals became more tumor-resistant; 3 animals survived past day 80 (34 days after a second inoculation with 2.5×10³ TA3Ha cells), and 2 animals survived past day 150 and showed no sign of tumor occurrence. Thus, the 2 surviving animals became completely refractory to inoculation with highly malignant TA3Ha cells.

Mouse Immune Response to Active Immunization With A-OSM/Ribi or A-BSM/Ribi

Immune response was evaluated in terms of antibody titer and lymphocyte proliferation to immunogen. Both IgG and IgM titers directed to A-BSM and A-OSM were clearly detectable. There was no significant difference between groups immunized subcutaneously and intraperitoneally. The determination was made after two immunizations with A-OSM/Ribi or A-BSM/Ribi as described above. There was no difference in terms of antibody titer, nor IgG vs. IgM titer response, between immunization with A-OSM vs. A-BSM.

To determine lymphocyte proliferation, mice were immunized twice subcutaneously with Ribi alone, A-OSM/Ribi or core protein from OSM/Ribi. Core protein was prepared by deglycosylation of OSM by trifluoromethanesulfonic acid according to the method described in Woodward et al. (Biochem., 26:5315-5322, 1987). That procedure results in loss of 80-90% of Tn antigen activity. Inguinal (regional) lymph nodes were excised and lymphocytes were cultured in the presence of A-OSM. After 5 days, [³H]thymidine (0.5 μCi) was added to each lymphocyte culture and radiolabel incorporation was measured. Interestingly, incorporation was stimulated significantly when the lymphocytes originated from mice immunized with A-OSM. Ribi alone or OSM core protein alone did not induce lymphocyte proliferation. That indicates regional lymph node lymphocytes of immunized animals are primed by a carbohydrate antigen, but not by core protein or Ribi adjuvant alone.

Lymphocyte proliferation induced by asialo-mucin was analyzed further by differential determination of B vs. T cells by passage through anti-B cell column and observation of T cell response. Response of purified T cells was significantly stimulated by A-BSM; however, the level of response in the purified T cell population was lower than that of total lymphocytes, i.e., the mixed preparation of B and T cells. That was due partially to elimination of adherent cells together with B cells, and partially to dilution of T cells. Ribi alone did not stimulate either B or T lymphocytes. The fact that T cell proliferation was greatly stimulated by A-OSM is demonstrated clearly by the observed suppression of this response by anti-Thy1.2 antibody, which depletes T cell response. Further studies indicated that T cells stimulated by either A-BSM or A-OSM were mainly helper T cells. Apparently lymphocyte proliferative response to asialo-mucin, but not to core protein or Ribi, includes a T cell response.

The functional role of proliferated A-OSM-specific T cells was examined by measuring the capacity of those cells to secrete the lymphokine IL-2. Lymphocytes from mice immunized with A-OSM or irradiated TA3Ha tumor cells responded to in vitro A-OSM stimulation by secreting IL-2. However, stimulation with OSM core protein did not induce the cells to produce IL-2.

The findings indicate that desialylated animal mucins provide a basis for anticancer vaccine development when properly combined with a T cell stimulator such as Ribi adjuvant. It is important to note that immune response to OSM core protein was absent or undetectable with the present analytical method. Therefore, undesirable side effects of core protein need not be taken into consideration.

Another important finding of the study is that mucin by itself is incapable of inducing a T cell immune response for suppression of tumor growth; this response occurs only in combination with a proper carrier such as Ribi adjuvant.

Since Tn and sialyl-Tn antigens are relatively simple carbohydrate structures, methods for their synthesis are readily available. Assembly of multiple carbohydrate epitopes bound to appropriate structures and capable of priming T cell immune response is desirable in the present context. Recently, a vaccine engineering technique has been described based on multiple antigenic site assembly (e.g., Tang & Lu, Proc. Natl. Acad. Sci USA, 86:9084-9088, 1989). Using this technique, multiple T/Tni/sialyl Tn epitopes could be assembled on a highly branched peptide carrier linked to a specific epitope readily presented to and recognized by T lymphocytes.

### EXAMPLE 4

Chemical Synthesis of Tn and Sialyl-Tn Antigens

Conjugated with Carrier Macromolecules

The basic idea for the preparation of multivalent antigen systems is summarized in FIG. 1A, Scheme I, in which the use of lysyllysine as a core matrix bearing multiple antigens as dendritic arms constitutes an essential part of that scheme.

Sequential conjugation with lysyllysine, in which three amino acid groups are available as reactive ends, will generate 3ⁿ Tn antigen residues. These residues will be converted to sialyl-Tn (NeuAcα2→6GalNAcα1→R) by chemical or enzymatic sialylation. To avoid the multiple steps involved in chemical sialylation, enzymatic sialylation using CMP-NeuAc (cytidine-monophosphosialic acid) and 2→6sialyltransferase would be preferable. The conjugates will be used as the immunizing antigens after conjugation to a carrier protein.

Synthesis of a multiple-antigen peptide system (MAP) using 1-butoxycarbonyl (Boc) β-Ala-OCH₂-Pam resin and lysine core was previously described (Tam, Proc. Natl. Acad. Sci. USA 85:5409-5413, 1988).

Synthesis of trivalent conjugates is accomplished by the coupling of the N-hydroxysuccinimyl derivative of Tn antigen with lysyllysine. The complete reaction sequence is shown in FIG. 1B, Scheme II.

The antigen 1 is synthesized according to published procedures (Paulsen & Holek, Carb. Res. 109:89-107, 1982; Grundler & Schmidt, Lieb. Ann. Chem. 1826-1847, 1984).

To eliminate the cationic nature of an amino group, which gives rise to highly charged conjugates, the amino group of serine needs to be modified by selective N-acetylation with acetic anhydride in methanol.

The resulting compound 2 is converted to its N-hydroxysuccinimide derivative in the presence of dicyclohexylcarbodiimide as a condensation agent. The coupling reaction of 3 with 4 (Bachem Bioscience Inc., Philadelphia, Pa.) is achieved by using a 4.5 M excess of 3, yielding trivalent conjugate 5. After purification by P2 column chromatography with H₂O, conjugate 5 is converted to its active ester 6 for further coupling with 4 or with a carrier protein.

### EXAMPLE 5

Synthetic Vaccines Based on Tn and Sialyl-Tn Antigens

The principle for making chemically unambiguous vaccines (summarized in FIGS. 2A-C) consists of: 1) synthesizing antigen clusters, 2) constructing multivalent systems and 3) configuring for effective presentation of synthetic antigens to an immune system.

Synthesis of Antigen Clusters by Tandem Linkage of Tn and Sialyl-Tn

Antigen clusters can be synthesized by tandem linkage of Tn and sialyl-Tn for use in active immunization, as shown in FIG. 2A. To eliminate the cationic nature of amino groups which gives rise to highly charged conjugates, the amino terminal of the cluster is modified preferably by acetylation. The carboxyl terminal of the cluster is linked to a spacer arm, e.g., 4-aminobutyric acid, to reduce interaction between epitopes and carrier molecules.

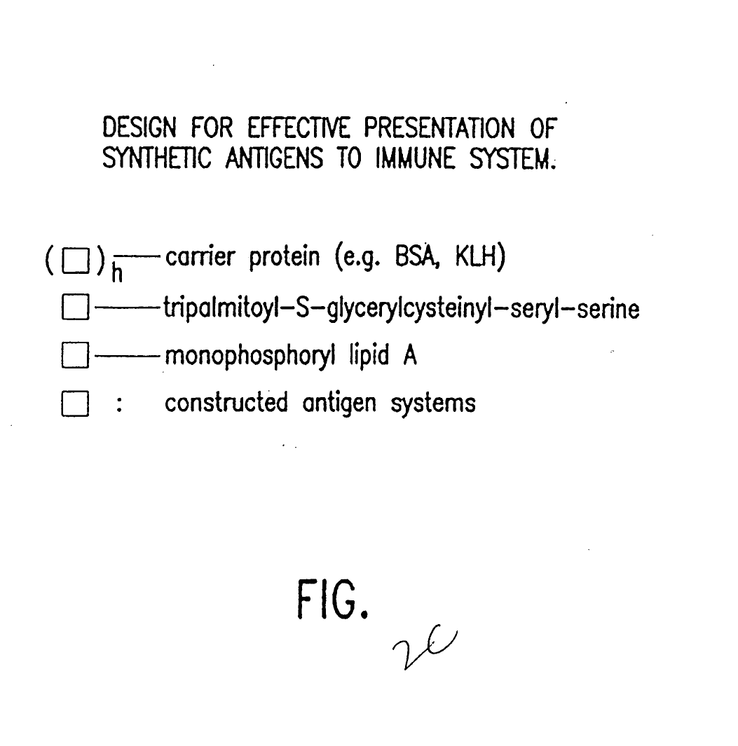

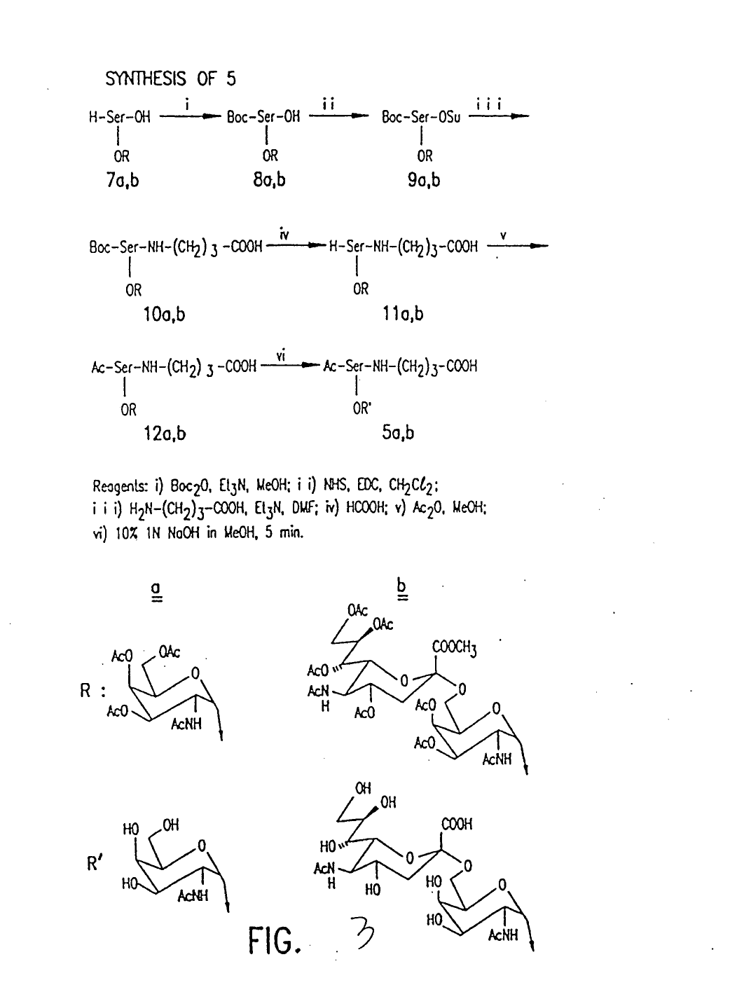

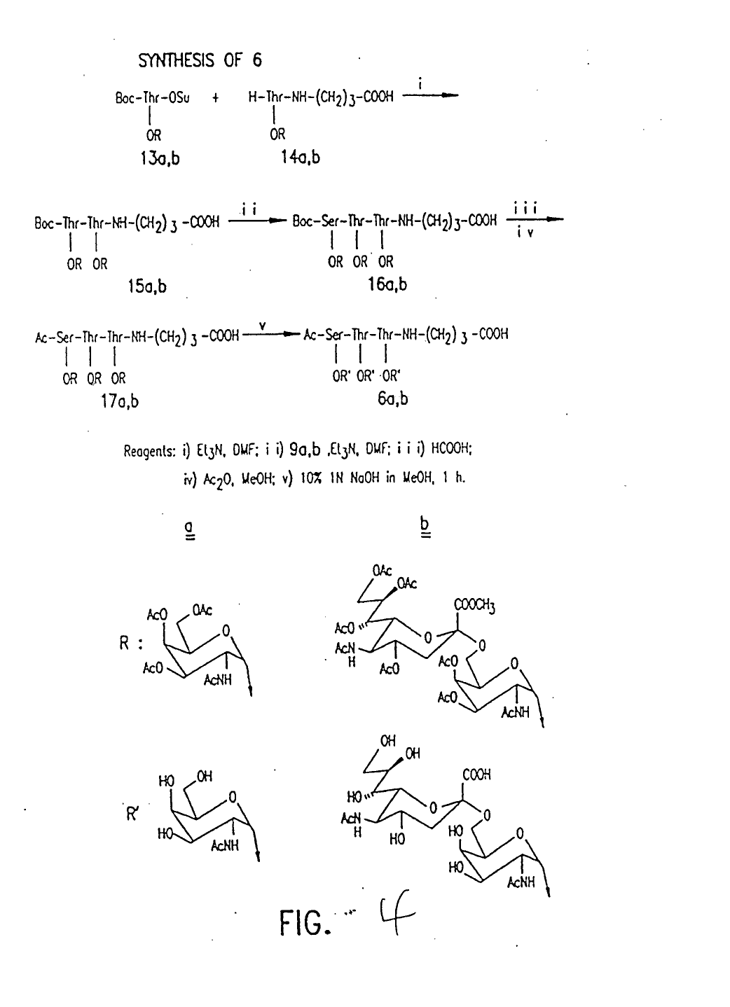

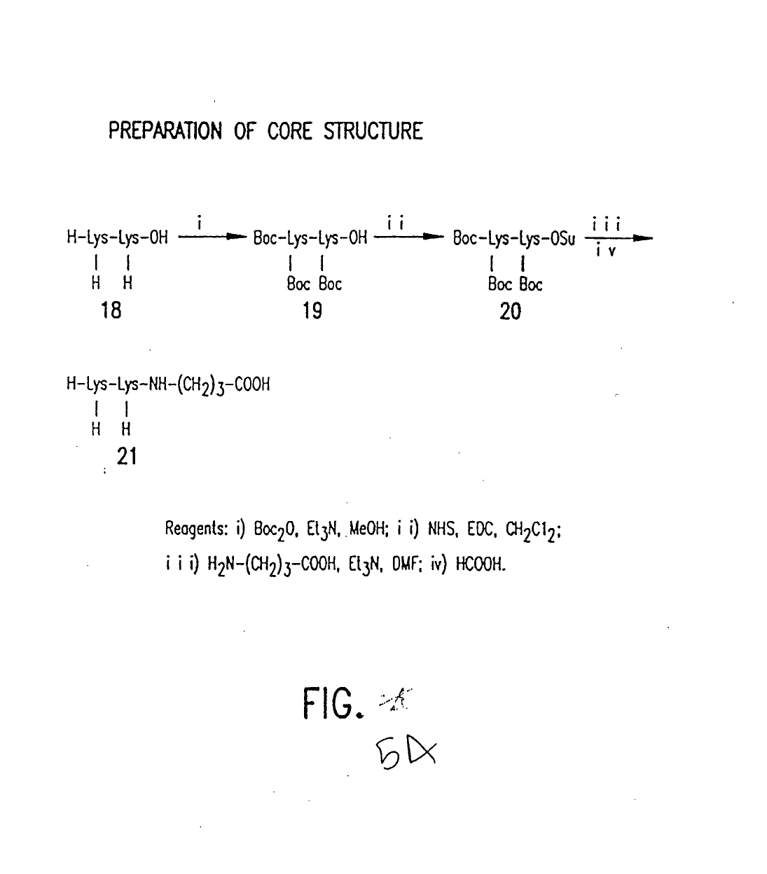

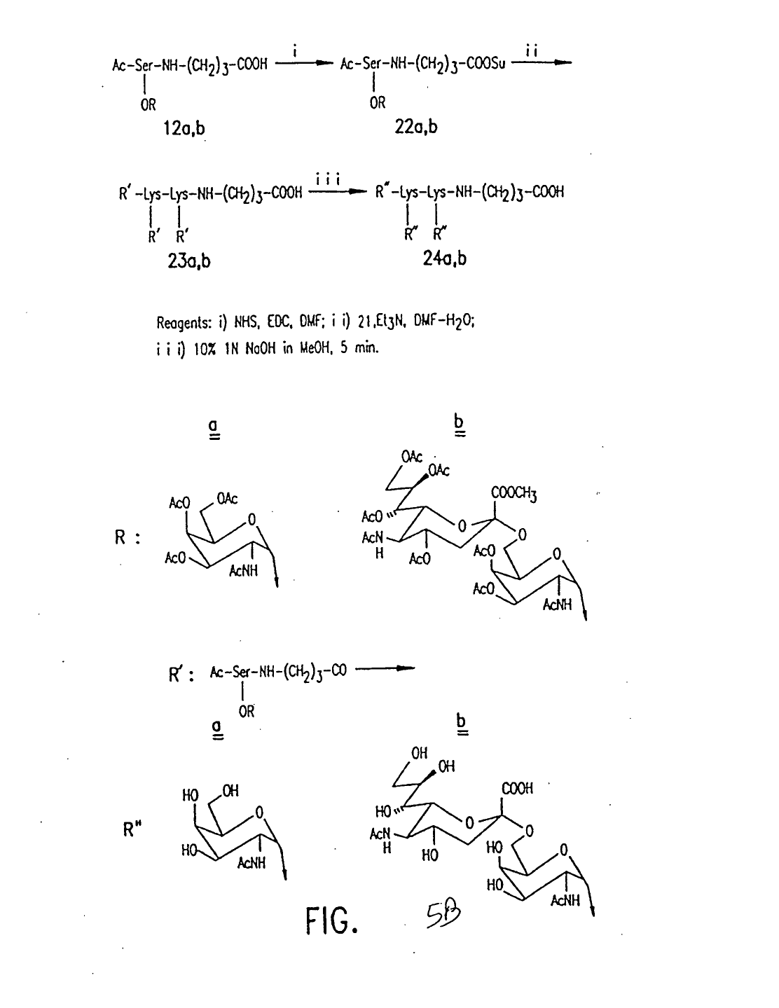

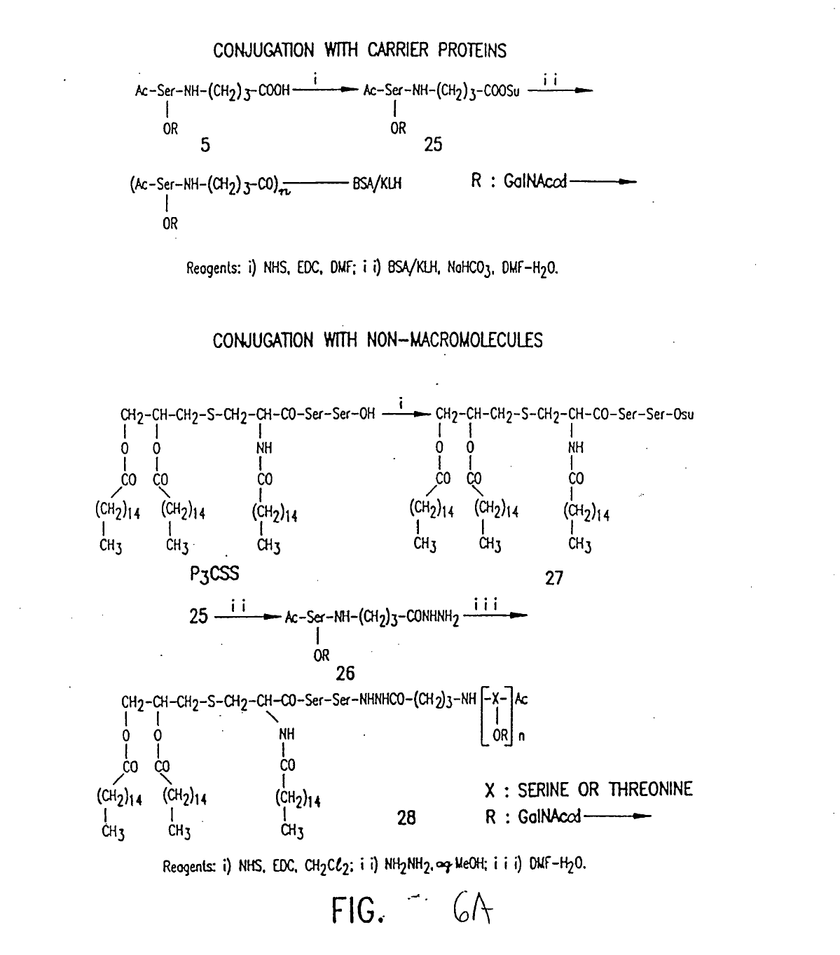

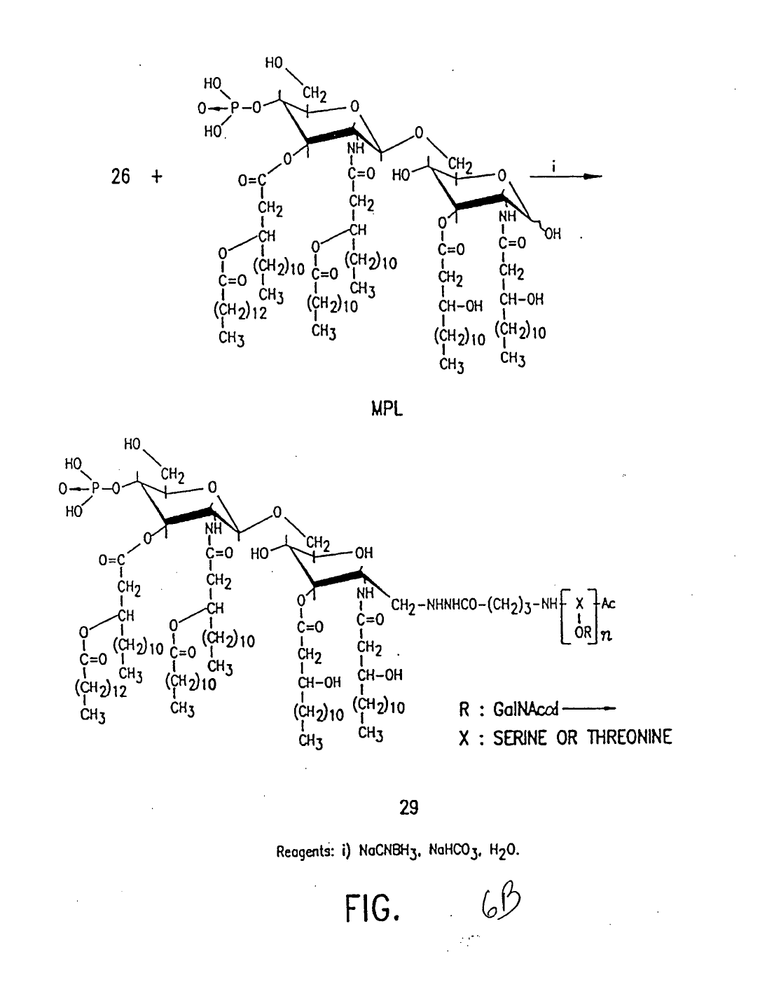

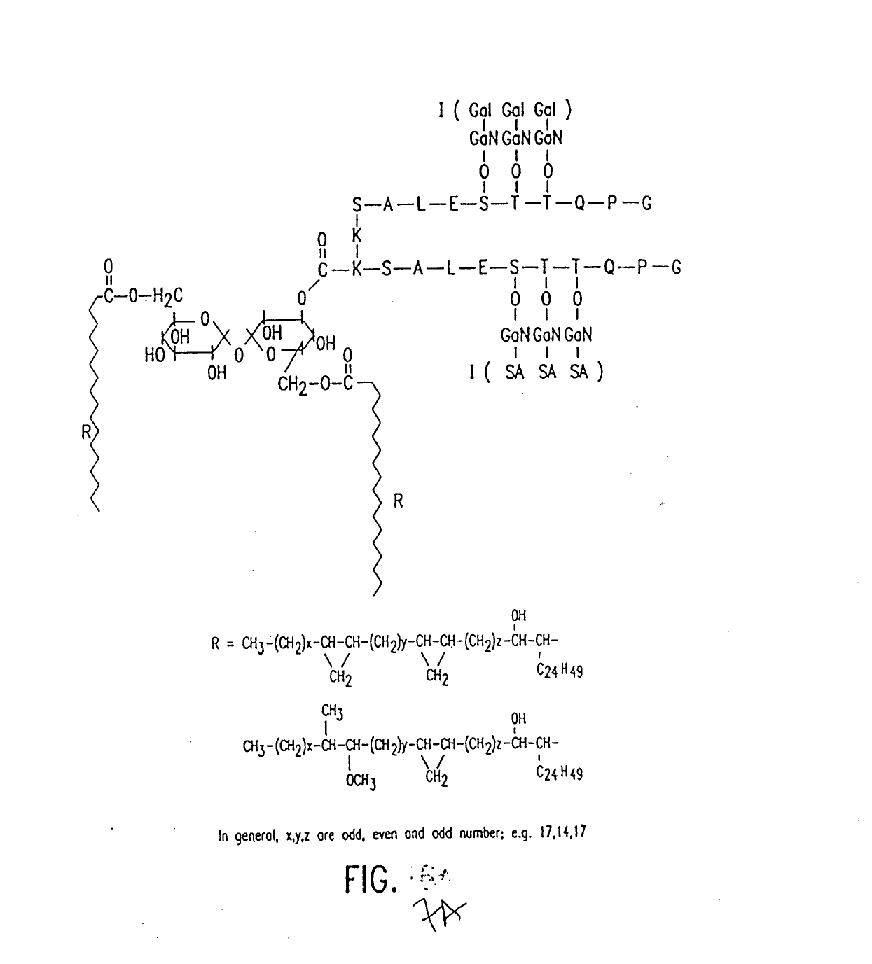

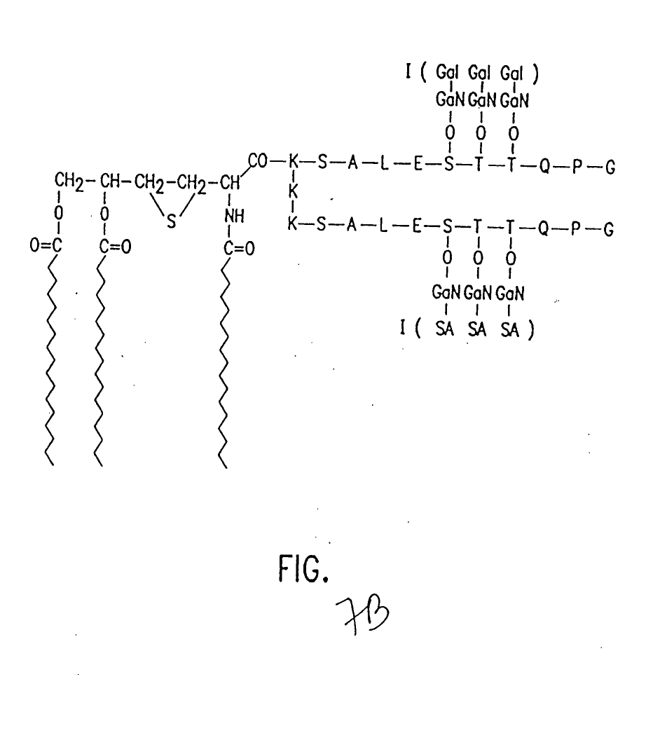

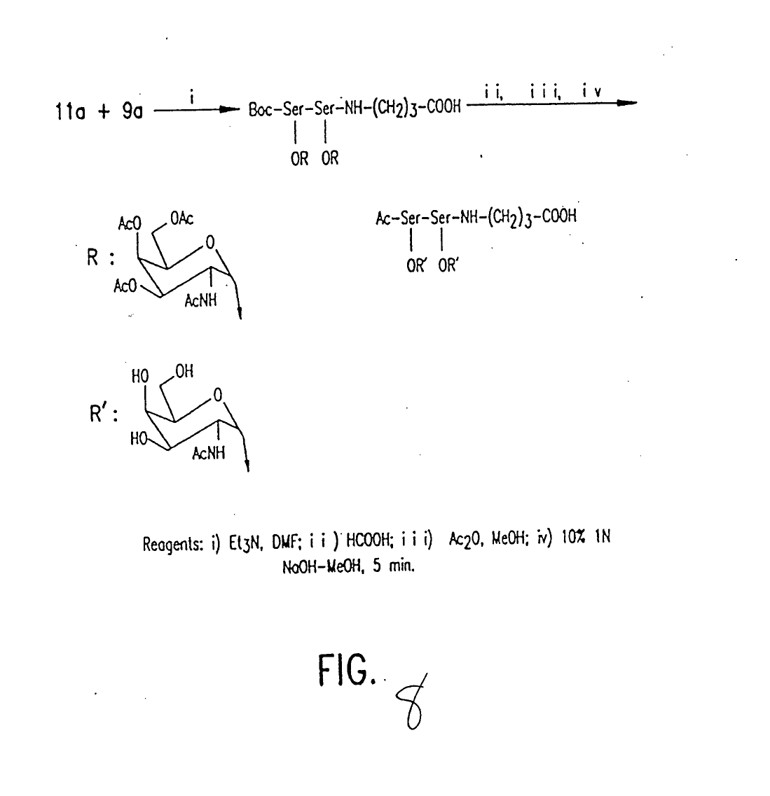

The complete reaction sequences for antigen clusters 5 and 6 of FIG. 2A-C are illustrated in FIGS. 3 and 4.

Synthesis of 5 (FIG. 3)

The starting compounds, 7a and 7b, are readily prepared according to published procedures (For 7a: Paulsen & Holek, Carb. Res., 109:89-107, 1982; Grundler & Schmidt, Liebigs. Ann. Chem., 1826-1847, 1984. For 7b: Iijima & Ogawa, Carb. Res., 172:183-193, 1988). Compound 7 is treated with di-tert-butyl decarbonate (Boc₂O) to give 8, which is then converted to its succinimide (Su) ester, 9, by treatment with N-hydroxysuccinimide (NHS) and 1-ethyl-3-(3-dimethyl-aminopropyl)-carbodiimide hydrochloride (EDC) in dry dichloromethane. Compound 9 is then coupled with 4-aminobutyric acid (Aldrich, Milwaukee, Wis.) in the presence of triethylamine (Et₃N) in dry N,N-dimethylformamide (DMF) giving 10. The amino protection is changed from Boc to acetyl (Ac) by treatment with formic acid (giving 11) followed by acetylation with acetic anhydride (Ac₂O) in methanol (MeOH). The resulting compound 12 is saponified to give 5 by rapid treatment (5 min) with 10% 1N sodium hydroxide (NaOH) in methanol.

Synthesis of Trivalent Conjugates

The complete reaction sequence is shown in FIGS. 5A and 5B and see Toyokuni et al., 198th ACS National Meeting, Miami Beach, Fla., September 1989; Abstr. CARB 51; Toyokuni et al., Tetra. Lett. 31:2673-2676, 1990). First, the core structure 21 is synthesized. L-lysyl-L-lysine 18 (Bachem Bioscience Inc., Philadelphia, Pa.) is treated with Boc₂O to give 19, which is subsequently converted to its active ester 20 by reaction with NHS and EDC in dry DMF. The coupling reaction of 20 with 4-aminobutryic acid followed by acid treatment yields 21.

Next, compound 12 (FIG. 5B) is activated by formation of its N-hydroxysuccinimide ester 22 and then coupled with 21 to give 23. Saponification of 23 with 10% 1N NaOH in methanol gives trivalent conjugate 24.

Sialyl-Tn conjugate 24b is prepared by enzymatic sialylation of 24a as described above.

Design for the Effective Presentation of Synthetic Antigens to the Immune System

To obtain an effective immune response, synthesized Tn/sialyl-Tn epitopes must be presented appropriately to the immune system. One of the goals in development of synthetic vaccines is the design of vaccines which do not require carrier proteins.

Recently, synthetic viral proteins covalently linked to tripalmitoyl-S-glycerylcysteinyl-seryl-serine (P₃CSS) have been shown to efficiently prime influenza virus-specific cytotoxic T lymphocytes (CTL) in vivo (Deres et al., Nature 342:561-564, 1989).

Therefore, besides being coupled to carrier proteins including bovine serum albumin (BSA) and keyhole-limpet hemocyanin (KLH), synthetic antigens are coupled to P₃CSS and to monophosphoryl lipid A, also known as Ribi adjuvant, (MPL), after minor modifications. The concept is illustrated schematically in FIGS. 6A and 6B wherein compound 5 is used as the synthetic antigen.

Coupling of 5 with BSA/KLH

Compound 5 is converted to its active ester 25 by treatment with NHS and EDC in DMF which is then coupled with BSA or KLH at pH 7-8 (NaHCO₃).

The coupling yield is determined by comparing the presence of free amino groups in KLH before and after the reaction using the 2,4,6-trinitrobenzensulfonic acid (TNBS) method (Snyder & Sobocinski, Anal. Biochem. 64:284-288, 1975).

Coupling of 5 with P₃CSS

Compound 25 is treated with hydrazine in aqueous methanol to give a hydrazide 26. The hydrazide is then coupled to the N-hydroxysuccinimide ester of P₃CSS 27 to give the conjugate 28.

Coupling of 5 with MPL (FIG. 6B)

The hydrazide 26 is reacted with MPL in the presence of sodium cyanoborohydride at pH 6, giving the conjugate 29.

T/Tn/Sialyl-Tn Covalently Linked to Lipid Adjuvant

Another design of a vaccine for stimulation of the immune response based on the fact that T/Tn/sialyl-Tn antigens themselves may not stimulate effectively an immune response but require a suitable lipid carrier such as Ribi adjuvant or P₃CSS as described above. Thus T/Tn/sialyl-Tn may be linked covalently to lipid adjuvant as shown in FIGS. 7A and 7B. Trehalose dimycolate will be coupled to peptides containing multiple STT sequences to which T/Tn/sialyl-Tn are coupled (FIG. 7A). Similarly, T/Tn/sialyl Tn can be linked covalently to P₃CSS as above (FIG. 7B). The constructs may serve as potential anti-cancer vaccines.

Monomeric synthetic Tn antigen coupled to KLH as described above was used to determine its effectiveness as a tumor vaccine. Mice were preimmunized at day −7 with 50 μg of synthetic Tn/Ribi, s.c. and were challenged with TA3Ha cells, i.p. at day 0. Mice were given cyclophosphamide on day 1 and antigen/Ribi on days 2, 5 and 12. Animals receiving CP alone or CP plus KLH/Ribi did not survive tumor challenge. However, 60% of mice receiving CP plus Tn-KLH/Ribi survived beyond 40 days of tumor challenge.

High anti-Tn titers were observed in mice after 2 s.c. immunizations with Tn-KLH/Ribi. The assay for anti-Tn antibodies was an ELISA configured after the RIA described above. Secondary antibodies to mouse IgG and IgM were conjugated with horseradish peroxidase and bound conjugate was exposed by reaction with o-phenylenediamine and hydrogen peroxidase. The colorimetric reaction was monitored at 492 nm. (Because of the monomeric nature of synthetic Tn antigen, most of the serum anti-Tn titer was of IgM type.)

The antibodies next were tested for the ability to bind to cells. A standard immunofluorescence protocol was followed (for example, see Selected Methods in Cellular Immunology, Mishell & Shiigi, eds. Freeman, SF, 1980). Briefly, cells were incubated with immune serum for about 30 minutes on ice. The cells were washed and exposed to fluorochrome conjugated anti-mouse Ig antibody. The cells were incubated for about 30 minutes on ice and then washed. Slides were prepared and fluorescence assessed in a fluorescence microscope. Immune sera were tested with TA3Ha cells. A comparable binding was seen in sera from synthetic Tn or A-OSM or A-BSM immunized mice.

The effectiveness of synthetic monomeric and dimeric Tn antigen was compared. Dimeric Tn antigen was prepared in the following manner. Compounds 11a and 9a were coupled by a series of treatments comprising triethanolamine in DMF, HCOOH, Ac₂O in methanol and 10% of 1N NaOH in methanol (FIG. 8). The dimeric Tn then was converted to the corresponding h-hydroxysuccinamide ester and conjugated to KLH.

Mice were immunized with 30 μg of monomeric Tn-KLH/Ribi or dimeric Tn-KLH/Ribi on days −7, 2, 5, 12 and 19. Monomeric Tn at the lower dose of 30 μg per injection (50 μg were used in the experiment described above) was not effective in preventing tumor challenge. However, dimeric Tn at this dosage was effective in preventing tumors in 40% of mice beyond 40 days after tumor challenge (FIG. 31). A reasonable conclusion is that the dimeric antigen elicited a higher immune response than did monomeric antigen. It is highly conceivable that polymeric Tn antigen with a higher valency synthesized by the methods described above will be much more effective than monomeric or dimeric Tn antigen.

### EXAMPLE 6

Active Immunization With Lactone

To determine the effect of B16 melanoma cell growth by active immunization of mice with GM₃ lactone or GM₃ coated on acid-treated Salmonella minnesota the following experiments were carried out. Ten BALB/c mice were immunized with native GM₃ or GM₃ lactone coated on acid-treated Salmonella minnesota. Immunization was carried out by intravenous injection of 200 μl of the GM₃ or GM₃ lactone preparation once per week for 4 weeks. Subsequently, 1.0×10⁵ B16 melanoma cells of clones F-1 or F-10, were subcutaneously injected into the back of the mice and tumor growth was observed after 20 days. As controls, other glycolipids, such as paragloboside coated on acid-treated Salmonella minnesota, and Salmonella minnesota alone, were used in the same amounts as discussed above. The results are shown in the table below.

TABLE
Effect of Immunization with GM₃ Lactone
on B16 Melanoma Development

| | Salmonella | GM₃ Adsorbed | GM₃ Lactone Adsorbed |
|---|---|---|---|
| | minnesota | on Salmonella | on Salmonella |
| Melanoma B16 | alone | minnesota | minnesota |
| F-1 | 10/10 | 10/10 | 2/10 |
| F-10 | 10/10 | 10/10 | 3/10 |

In the table, the numbers indicate the number of animal which died over the total number of animals immunized. The results in the table above demonstrate that tumor growth was reduced in the group immunized with GM₃ lactone but not in the group immunized with GM₃ or with other glycolipids, such as paragloboside coated on Salmonella Minnesota, or with Salmonella minnesota alone. These results demonstrate that GM₃ lactone but not GM₃ is capable of suppressing tumor growth in vivo.

### EXAMPLE 7

Assembly of Tn or sialyl-Tn Antigen

Synthetic Tn or sialyl-Tn antigen can be assembled in tandem repeat form, for example, to Ser-Thr-Thr, or to another order of the hydroxyamino acids (as shown in FIG. 9A). Synthetic Tn or sialyl-Tn antigen also can be tandemly assembled on any synthetic compound R which is tandemly linked through a spacer “X” with a terminal R group linked to a functional group “Z” which is ready to couple to a carrier molecule. The distance between R groups and the orientation of α-GalNAc or NeuAcα2→6GalNAcα is similar to that observed in Ser-Thr-Thr or another order of those hydroxyamino acids (FIG. 9B). The optimal number of Tn or sialyl-Tn residues appears to be about 3, derived on an empirical basis from many experiments.

The tandem repeats of Tn or sialyl-Tn can be used as the functional antigen unit. That antigen unit can be bound to various types of carrier molecules, such as, protein, lipid or peptide, particularly those having binding ability to MHC class I or class II proteins, and particularly class II proteins.

### EXAMPLE 8

Selection of Peptide Mimetics Having the Same Surface Profile as Tumor-Associated Carbohydrate Antigens

Peptides having a surface profile complementary to a defined tumor-associated carbohydrate antigen can be selected by use of monoclonal antibody directed to the carbohydrate antigen. Complementary peptides can be selected from a phage display random peptide library. Anti-Tn monoclonal antibody (CU1, an IgG3 antibody) or anti-sialyl-Tn monoclonal antibody (TKH1, an IgG1 antibody) are coated on a plastic surface. Phage bearing randomly synthesized 8-mer to 15-mer peptides (5×10⁷ to 10⁸ phages per experiment) are added and peptides having preferential binding to anti-Tn or anti-sialyl-Tn antibody are selected. The principle of the selection cycle is illustrated in FIG. 10. Selected peptides should have the same surface profile as Tn or sialyl-Tn carbohydrate antigen, even though it is composed of amino acids. Because the conformational structure of some peptides may be unstable, stabilization of the peptides using known chemistries may be necessary. Mimetics thus obtained are capable of inhibiting binding of anti-Tn or anti-sialyl-Tn antibody to tumor cell surface antigens. Such mimetics, if linked to a proper carrier, should be strong immunogens and should generate an IgG response or a T cell response.

### EXAMPLE 9

Selection of Peptide or Peptides Having Specific Sequence with Capability to Bind to MHC Class II or Class I Proteins: Use of Such Peptides as Carriers of Tumor-Associated Carbohydrate Antigens to Induce IgG or T Cell Response

To promote helper T cell (CD4)-dependent production of IgG antibody directed to a defined carbohydrate antigen, the antigen should be presented through MHC class II proteins to the TCR/CD3 complex. Signaling through that presentation induces proliferation of CD4 cells and in IL-2 production leading to B cell proliferation and antibody production. The key mechanism is binding of processed peptide from exogenous protein antigen to MHC class II proteins in the peptide binding groove (see FIG. 11A and FIG. 12B). Such peptides having a specific binding surface profile complementary to MHC class II proteins are an excellent carrier for tumor-associated carbohydrate antigens such as Tn and sialyl-Tn, if the antigens are bound properly to the peptide. To identify and produce significant amount of such peptide, again, a phage display random peptide library can be used. A mixture of recombinant MHC class II proteins, particularly representing a domain containing a peptide binding groove, are coated on a plastic surface, and selection of peptides is made. MHC class II could be, for example, DR β1 or β2, since the majority of the human population carries those molecular species. Specific binding of the selected peptide to MHC class II DR β1 or β2 is verified, the peptide is stabilized properly and used as a carrier for carbohydrate antigens, particularly Tn and sialyl-Tn. Alternatively, peptide mimetics of Tn and sialyl-Tn, selected and established as described in Example 8, are bound to such carrier peptides.

Selection of peptide mimetics of Tn and sialyl-Tn, and selection of peptides having binding ability to MHC class II DR β1 or β2 are made by a combination of procedures as

What is claimed is:

1. A vaccine for preventing or retarding growth and replication of cancer cells that express a carbohydrate antigen, said vaccine comprising:
(a) a pharmaceutically effective amount of said carbohydrate antigen, or a mimetic of said carbohydrate antigen, wherein said carbohydrate antigen comprises Tn or sialyl Tn; and
(b) a pharmaceutically acceptable carrier.

2. The vaccine of claim 1, wherein said vaccine comprises a molecule comprising multivalent Tn or sialyl-Tn epitopes.


SEQUENCE LISTING (6 sequences)

SEQ ID NO: 1 - protein, 8 aa, Artificial Sequence
  Description of Artificial Sequence Illustrative inhibitory peptide
APWLYGPA

SEQ ID NO: 2 - protein, 9 aa, Vesicular stomatitis virus
FAPGNYPAL

SEQ ID NO: 3 - protein, 8 aa, Sendai virus
RGYVYQGL

SEQ ID NO: 4 - protein, 9 aa, Influenza A virus
ASNENMETM

SEQ ID NO: 5 - protein, 10 aa, Unknown Organism
  Description of Unknown Organism Adenovirus
SGPSNTPPEI

SEQ ID NO: 6 - protein, 22 aa, Artificial Sequence
  Description of Artificial Sequence Synthetic peptide
GPQTTSELASKKSALESTTQPG